(19) United States
(12) Patent Application Publication
(10) Pub. No.: US 2025/0154592 A1
(43) Pub. Date: May 15, 2025

(54) Methods and Compositions for Evaluating Biomarkers in Salivary Exosomes and Evaluating Cognitive Fatigue

(71) Applicants: The Regents of the University of California, Oakland, CA (US); The United States Government as Represented by the Department of Veterans Affairs, Washington, DC (US)

(72) Inventors: Varghese John, Los Angeles, CA (US); Bruce Teter, Los Angeles, CA (US); Samuel Whitaker Johnson-Cohn, Los Angeles, CA (US)

(21) Appl. No.: 18/839,181

(22) PCT Filed: Feb. 20, 2023
(86) PCT No.: PCT/US2023/062888

§ 371 Date: Aug. 16, 2024

Related U.S. Application Data
(60) Provisional application No. 63/313,717, filed on Feb. 24, 2022.

(51) Int. Cl. C12Q 1/6883; C12Q 1/686; G01N 33/543; G01N 33/573; G01N 33/68

(52) U.S. Cl. CPC C12Q 1/6883; C12Q 1/686; G01N 33/54388; G01N 33/573; G01N 33/6896; C12Q 2600/178; G01N 2333/4704; G01N 2333/91225; G01N 2333/926; G01N 2333/948; G01N 2333/988; G01N 2800/2814

(57) ABSTRACT

Disclosed herein are methods and compositions for diagnosing cognitive fatigue. Specifically, the disclosure provides a method of diagnosing a subject as suffering from cognitive fatigue, which comprises measuring the amount of a biomarker, comprising phosphoglycerate kinase 1 (PGK1) and/or miR3185, in an exosome sample obtained from a saliva sample from the subject, comparing the amount with a control, and identifying the subject as suffering from cognitive fatigue.

### CROSS-REFERENCE TO RELATED APPLICATIONS

This application claims the benefit of U.S. Patent Application No. 63/313,717, filed Feb. 24, 2022, which is herein incorporated by reference in its entirety.

### ACKNOWLEDGEMENT OF GOVERNMENT SUPPORT

This invention was made with government support under Grant Number FA9550-17-1-0399, awarded by the U.S. Air Force, Office of Scientific Research. The Government has certain rights in the invention.

### NO SEQUENCE LISTING

No sequence listing is required because no sequences are explicitly enumerated herein.

### BACKGROUND OF THE INVENTION

### 1. Field of the Invention

The field of the invention generally relates to diagnostics for cognitive fatigue.

### 2. Description of the Related Art

Fatigue is known to impair cognitive performance, resulting in what is referred to as cognitive fatigue (CF). CF causes serious performance dysfunction in many professions, e.g., medical and airline professions. CF is characterized by an inability to maintain optimal performance during prolonged cognitive effort; it can manifest after long-duration cognitive activity, sleep deprivation or excessive exercise. Determining the extent of CF for an individual has important implications for high-risk jobs, including medical doctors and airplane pilots; and the ability to assess CF would be a useful tool in the prevention of catastrophic accidents. Typically, the extent of fatigue and thus risk for CF is determined by self-reported assessments which can prove to be subjective and unreliable. Thus, a method for objectively assessing CF would be of great value and identification of biomarkers associated with fatigue has the potential to be a first step in development of a rapid antigen detection-based assay to determine the risk for CF.

### SUMMARY OF THE INVENTION

In some embodiments, the present invention is directed to methods of A method of evaluating expression levels of PGK1 and miR3185 (preferably hsa-miR-3185) in a subject, which consists of: obtaining an exosome sample from a saliva sample from the subject, measuring the amount of the biomarker in the exosome sample, and optionally measuring in the exosome sample the amount of one or more additional biomarkers selected from the group consisting of: LEG1, AMY1A, BPIFA2, CA6, DPP4, DMBT1, hsa-miR-518e-3p, hsa-miR-182-5p, hsa-miR-614, hsa-miR-1296-3p, hsa-miR-126-3p, hsa-miR-1257, hsa-miR-134-3p, hsa-miR-105-5p, hsa-miR-4536-5p, hsa-miR-642a-5p, and hsa-miR-140-5p.

In some embodiments, the present invention is directed to methods of preparing an exosome sample for measuring the amount of a biomarker, preferably PGK1 and/or miR3185, therein, which comprises obtaining a saliva sample from one or more subjects, adding a protease inhibitor to the saliva sample, removing solids in the saliva sample to obtain a supernatant, and isolating exosomes present in the supernatant on a substrate surface.

In some embodiments, the present invention is directed to methods of measuring expression levels of a biomarker, preferably PGK1 and/or miR3185, in an exosome sample obtained from a saliva sample, which comprises obtaining an exosome sample that has been prepared as described herein, and measuring the amount of the biomarker.

In some embodiments, the present invention is directed to methods of evaluating expression levels of a biomarker, preferably PGK1 and/or miR3185, associated with cognitive fatigue in a subject, which comprises obtaining an exosome sample from a saliva sample from the subject and measuring the amount of the biomarker in the exosome sample.

In some embodiments, the present invention is directed to methods of diagnosing a subject as suffering from cognitive fatigue, which comprises measuring the amount of a biomarker, which is PGK1 and/or miR3185, in an exosome sample obtained from a saliva sample from the subject, comparing the amount with a control, and identifying the subject as suffering from cognitive fatigue where the measured amount of PGK1 is about 1.3-1.4 fold increase and/or the amount of miR3185 is about 1.2-1.3 decrease compared to the control.

In some embodiments, the methods further include measuring one or more additional biomarkers selected from the group consisting of: LEG1, AMY1A, BPIFA2, CA6, DPP4, DMBT1, and the microRNAs set forth in FIG. 25 and FIG. 26 (preferably hsa-miR-518e-3p, hsa-miR-182-5p, hsa-miR-614, hsa-miR-1296-3p, hsa-miR-126-3p, hsa-miR-1257, hsa-miR-134-3p, hsa-miR-105-5p, hsa-miR-4536-5p, hsa-miR-642a-5p, and hsa-miR-140-5p).

In some diagnostic methods, the subject is diagnosed as suffering from cognitive fatigue where: A) the amount of LEG1, AMY1A, BPIFA2, CA6, and/or DPP4 is increased, B) the amount of DMBT1 is decreased, or both A) and B) as compared to the control.

In some diagnostic methods, the subject is diagnosed as suffering from cognitive fatigue where: a) the amount of hsa-miR-1296-3p, hsa-miR-126-3p, hsa-miR-1257, hsa-miR-134-3p, hsa-miR-105-5p, hsa-miR-4536-5p, hsa-miR-642a-5p, and/or hsa-miR-140-5p, b) the amount of hsa-miR-518e-3p, hsa-miR-182-5p, and/or hsa-miR-614 is decreased, or both a) and b) as compared to the control.

In some diagnostic methods, the subject is diagnosed as suffering from cognitive fatigue where: 1) the amount of LEG1, AMY1A, BPIFA2, CA6, and/or DPP4 is increased, 2) the amount of DMBT1 is decreased, or both 1) and 2); and i) the amount of hsa-miR-1296-3p, hsa-miR-126-3p, hsa-miR-1257, hsa-miR-134-3p, hsa-miR-105-5p, hsa-miR-4536-5p, hsa-miR-642a-5p, and/or hsa-miR-140-5p, ii) the amount of hsa-miR-518e-3p, hsa-miR-182-5p, and/or hsa-miR-614 is decreased, or both i) and ii) as compared to the control.

In some embodiments, the exosome sample is prepared as described herein.

In some embodiments, the saliva sample is obtained from the subject or subjects after deliberately and consciously performing a cognitive activity for a given period of time with or without one or more breaks. In some embodiments, the given period of time is about 1 hour or more, about 2 hours or more, about 3 hours or more, about 4 hours or more, about 5 hours or more, about 6 hours or more, about 7 hours or more, about 8 hours or more, about 9 hours or more, about 10 hours or more, about 11 hours or more, about 12 hours or more, about 13 hours or more, about 14 hours or more, about 15 hours or more, about 16 hours or more, about 17 hours or more, about 18 hours or more, about 19 hours or more, about 20 hours or more, about 21 hours or more, about 22 hours or more, about 23 hours or more, or about 24 hours or more. In some embodiments, each break is independently about 5 minutes to about 45 minutes with the ratio of the sum amount of the one or more breaks to the given period of time being about 1:25 to about 1:8 or less.

In some embodiments, the biomarkers that are proteins are measured via a lateral flow assay device and/or the biomarkers that are microRNAs are measured via PCR.

In some embodiments, an amount of PGK1 that is about 1.3-1.4 fold more than a given control and/or the amount of miR3185 that is about 1.2-1.3 fold less than the given control results in a positive reading for cognitive fatigue.

In some embodiments, the biomarker, e.g., PGK1 and/or miR3185, is assayed using a lateral flow test (LFT) device.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1: Study flow scheme for Profile of Mood States (PoMS) assessment, sample collection, processing and analysis. (A) Resident participants (n=36) were enrolled. (B) Participants completed PoMS questionnaire and (C) collected saliva before a (D) 12-hour work shift. (E) Saliva was collected and the (F) PoMS questionnaire completed again after the work shift. (G) Total Mood Disturbance (TMD) and subscale (for example, fatigue-inertia, FI) scores were calculated. Isolation of exosomes comprised (H) centrifugation, (I) binding of exosomes to exosome marker-specific (and in one instance, neuronal-marker specific) antibody-conjugated Dynabeads, (J) release of exosomes for processing and Omics analyses. Based on TMD, participant samples were separated into Test (decrease in TMD or ‘improved’ mood), Discovery (increased in TMD or mood disturbance, and Validation (little/no change in TMD) groups. Exosomes from each group underwent the analyses shown.

FIG. 2-FIG. 5: Difference in Total Mood Disturbance (TMD) as assessed by the Profile of Mood States (PoMS) allows separation into Discovery, Validation, and Test groups. FIG. 2: The PoMS scores for anger-hostility (AH), confusion-bewilderment (CB), depression-dejection (DD), fatigue-inertia (FI), tension-anxiety (TA), and vigor-activity (VA) are shown pre- and post-work shift. Statistics performed using a Wilcoxon Signed Rank Test where CB *p=0.003, FI *p=0.0393, and VA *p=0.0047. In each set of bars, pre-work shift is the first bar and post-work shift is the second bar. FIG. 3: The combined TMD score for all participants pre-work shift (first bar) and post-work shift (second bar) is shown. Statistical analyses performed using the Wilcoxon Signed Rank Test where *p=0.0200. FIG. 4: The number of participants sorted into each group (x-axis) and the difference between pre- and post-work shift TMD (y-axis) is shown. The 6 participants with the greatest decrease in TMD score are in the Test group (Participant #'s 1-6), the 20 participants with the greatest increase in the Discovery group (Participant #'s 17-36), and 10 intermediate participants in the Validation group (Participant #'s 7-16). FIG. 5: The number of participants sorted into each group (x-axis) and the difference between pre- and post-work shift FI (y-axis) is shown. Test group=Participant #'s 1-6, Discovery group=Participant #'s 17-36, and Validation group=Participant #'s 7-16.

FIG. 6-FIG. 10: Multi-omics Analysis Reveals Quantifiable Differences in Protein and miRNA Abundances in the Test Group pan- and individual neuron-derived exosomes. FIG. 6: Differences in the abundances of proteins present in the salivary exosomes of Test group participants pre- and post-work shift are illustrated via volcano plot. The log₁₀ (abundance ratio: post-work/pre-work) is plotted against −log₁₀ (p-value). Decreased proteins are points≤0, Increased proteins are points >0. FIG. 7: A Venn diagram shows the overlap of proteins found in exosomes isolated with pan-exosomal or neuron-selective exosomal markers for a single participant. FIG. 8: Protein fold change for the single Test group participant is shown when using global proteomics on exosomes isolated by a pan-exosome set of antibodies (Global MS (exosome abs: CD9, CD63, CD81); first bars of each set) or targeted MS on 4 corresponding to the 4 proteins (BPIFA2, CSTB, PIGR, PKM) present in exosomes isolated using an antibody to a neuron-specific exosome marker (Targeted MS (neuronal ab: CD171); second bars of each set). FIG. 9: Differences in the abundances of miRNAs present in the salivary exosomes of participants pre- and post-work shift are illustrated via volcano plot. The log₁₀ (abundance ratio: post-work/pre-work) is plotted against −log₁₀ (p-value). Decreased miRNA are points≤0, Increased miRNA are points >0. FIG. 10: Fold change of miR-519d and miR-1296 as measured using the NanoString platform (y-axis) shows a moderate and strong positive correlation (miR-519d R²=0.4952; miR-1296 R²=0.9486) with fold change as measured by qPCR (x-axis). Top line is miR519d, bottom line is miR1296.

FIG. 11-FIG. 14: Interconnected Protein & miRNA Networks Regulate Molecular Pathways Associated with Increased TMD Score in the Discovery Group. FIG. 11: Differences in the abundances of proteins and miRNAs present in the salivary exosomes of Discovery group participants (n=20) pre- and post-work shift are illustrated via volcano plot; significant differences are points to the right of the vertical line marked p=0.05. The log₁₀ (abundance ratio: post-work/pre-work) is plotted against the −log₁₀ (p-value). Decreased proteins and miRNA are points≤0, Increased proteins and miRNA are points >0. FIG. 12: The mean fold change of three significantly altered miRNA (miR-3185, miR-642-5p, miR-134-3p) is shown to inversely relate to the abundances of proteins encoded by one of their target genes (PGK1, PIGR, YWHAZ). FIG. 13: Gene set enrichment analysis of upregulated and downregulated proteins using Enrichr (Chen et al., 2013) shows enrichment of KEGG database (Kanehisa & Goto, 2000) molecular pathways after a 12-hour work shift. Results ranked by p-value according to p-value. FIG. 14: Functional protein association network analysis using STRING (Szklarczyk et al., 2019) shows associated and interacting protein networks are differentially regulated after a 12-hour work shift.

FIG. 15: Identification of differentially abundant proteins and miRNA in salivary exosomes in the Discovery group. Six proteins were increased and 8 proteins decreased, with a fold-change greater than 1.2 fold. Three proteins are encoded by genes that could be a target of 3 of the miRNAs that changed in abundance in an opposite direction to that of the protein (as expected for the normal downregulation of gene expression by a miRNA); these 3 protein gene-miRNA pairs are shown in the same row.

FIG. 16-FIG. 18: Proteins & miRNA identified in the validation group associate with total mood disturbance, fatigue-inertia and work. FIG. 16: 12 of the 14 proteins identified as significantly altered in the discovery group were also identified in the Validation group. The abundance of each protein correlates with the fatigue-inertia (FI) PoMS subscale (PGK1), total mood disturbance PoMS category (SPRR3, PIGR, CSTB, FABP5, YWHAZ) or work in general (DPP4, BPIFA2, CA6, AMY1A, LEG1, and DMBT1, FABP5). FIG. 17: PGK1 abundance ratio (y-axis) shows a weak positive correlation (R2=0.3894) with FI subscale difference in validation group participants after a work shift. FIG. 18: The change in miR3185 levels (measured by qRT-PCR) for each participant from post-shift to pre-shift (post/pre; <1.0 values is a decrease in post-shift samples) are plotted versus the participant's POMS subscale Fatigue-Inertia difference (<0 values are a decrease in post-shift). The correlation coefficient was R2=0.53.

FIG. 19-FIG. 21: Integration of test, discovery and validation group PGK1 and miR3185 abundances displays trends indicative of biomarker potential. FIG. 19: Bar graph comparing PGK1 fold change from Test and Discovery group subjects in which PGK1 was identified. The Test group was less fatigued after work, while the Discovery group was more fatigued. FIG. 20: The change in miR3185 levels (measured by qRT-PCR) for subjects in the Discovery and Validation group plotted versus the PGK1 abundance ratios in saliva of subjects where it was identified. The correlation coefficient was R2=0.3553. FIG. 21: The bar graph comparing average PGK1 abundances in the Discovery group participants pre- and post-work showing significant difference where *p<. 05. The dotted line represents an amount of PGK1 abundance, above which, participants are likely to experience cognitive fatigue.

FIG. 22: Difference in Total Mood Disturbance (TMD) Positively Correlates with Difference in Fatigue-Inertia (FI) Subscale as assessed by the Profile of Mood States (PoMS). The difference in subject FI score in PoMS (y-axis) is shows a correlation (R²=0.646) with difference in subject TMD score (x-axis).

FIG. 23: Analysis of PoMS scores pre- and post-work shift.

FIG. 24: Test Group pan-exosome marker immunoprecipitated proteins of interest and corresponding miRNA that may regulate the gene encoding the protein.

FIG. 25: Discovery group miRNAs that change with PoMS TMD.

FIG. 26: Discovery group miRNAs that change with PoMS subscale FI.

### DETAILED DESCRIPTION OF THE INVENTION

Disclosed herein are methods and compositions for the non-invasive detection of cognitive fatigue in subjects.

Fatigue-associated changes in overall physical state such as dryness of the mouth may be, in part, due to biochemical changes in cellular signaling processes and the molecular composition of saliva. While saliva can be obtained easily and non-invasively in sufficient quantities for analyses, the ability to identify biomarkers of fatigue is hindered by the immense complexity and dynamic range of the of the salivary proteome and transcriptome. Here, the challenges caused by the complexity and dynamic range of the of the salivary proteome and transcriptome are reduced by assaying biomarkers in isolated exosomes, a subset of extracellular vesicles (EVs), in saliva.

Exosomes are small, 50-150 nm diameter, particles comprised of a lipid bilayer that carry cargo in their interior or on their surface. The surface of exosomes is enriched in tetraspanin marker proteins CD9, CD63, and CD81 important for the organization of membrane domains. Exosomes also contain tumor susceptibility gene 101 (Tsg101) and ALG-2-interacting protein X (ALIX) that are part of the Endosomal Sorting Complexes Required for Transport (ESCRT) machinery involved in intracellular vesicle formation and sorting of cargo. In a process still being elucidated, during formation exosomes are loaded with cytoplasmic proteins, nucleic acids including microRNAs, and membrane constituents that reflect the parent cell's biochemistry. Exosomes are released from all cell types including neurons and can act as intercellular signal carriers. Sleep deprivation is known to be associated with a decline in cognitive function and alterations in levels of intracellular proteins and nucleic acids, as well as circulating signaling molecules. Thus, exosomes may carry molecular signals reflective of changes in the physiology of the central nervous system (CNS) associated with the onset of fatigue-associated cognitive impairment.

The potential for exosomes in saliva to reflect changes in cognitive function is due to the presence of anatomical connections from the CNS, specifically innervation of Cranial Nerves VII and IX from the superior and inferior salivary nuclei to the oral cavity and the parotid and submandibular glands, or directly from blood through the vasculature in the oral cavity. The hypothesis that salivary exosomes carry signals that influence and/or are indicative of the changes in brain function is supported by several reports on salivary exosome proteins changing with HIV-associated cognitive deficits, in traumatic brain injury concussion-related cognitive fatigue, and in monozygotic twins discordant for chronic fatigue syndrome. Further, proteins such as beta-amyloid and tau, implicated in the impaired cognitive function associated with Alzheimer's disease, change in the total saliva protein pool and are present in bloodborne brain-derived EVs. These findings make it reasonable to hypothesize that other proteins and biomarkers related to cognitive function may be contained in salivary exosomes.

Here, to test the hypothesis that exosomes originating from cells of the CNS/brain are present in saliva and reflect physiological states such as fatigue, saliva was collected from medical resident participants before and after a 12-hour work shift and isolated exosomes for analysis. Participants were asked to complete a modified Profile of Mood States (PoMS) questionnaire both before and after their work shift, and the changes in subscale scores for self-assessed mood states such as fatigue were used to generate Total Mood Disturbance (TMD) scores. The isolated exosomes were subjected to multi-omics analyses, including quantitative proteomics and miR-omics, to identify salivary exosome-borne proteins and microRNAs associated with changes in TMD scores.

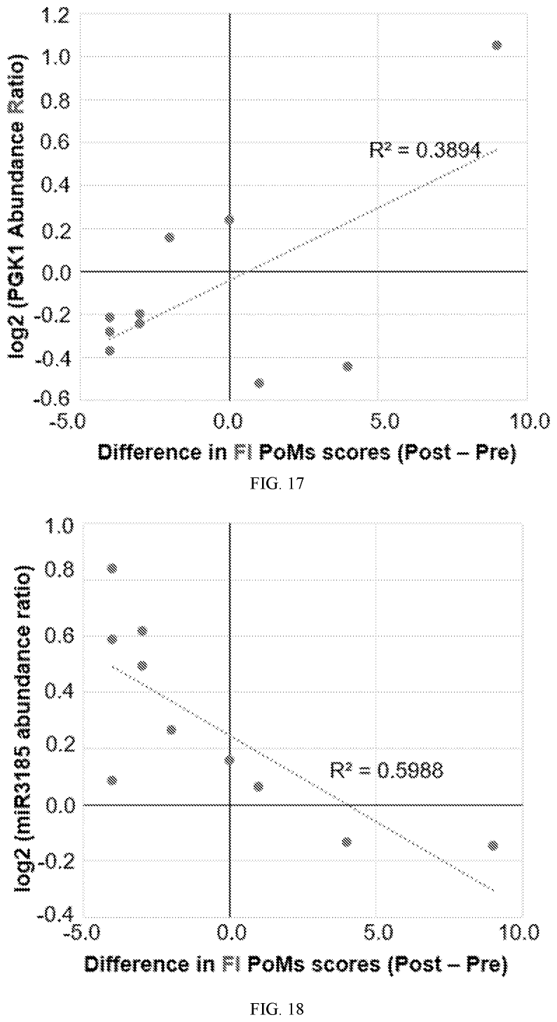

Proteomics analysis of Test group saliva confirmed successful enrichment of exosome populations and identified changes in the abundance several proteins. Some of the proteins with altered abundances are known to be associated with Chronic Fatigue Syndrome (CFS), including alpha amylase 1 (AMY1A), cystatin-B (CSTB), polymeric immunoglobulin receptor (PIGR), deleted in malignant brain tumors 1 protein (DMBT1), lysozyme C (LYZ) and ras-related C3 botulinum toxin substrate 1 (RAC1). The abundance of these CFS-associated proteins increased in some instances and decreased in others, without correlation to the ‘improved’ mood reported by the Test group. It is significant that two biomarkers, protein PGK1 and miR3185 show abundance change directions that switch when the correlated mood state switches from negative to positive (FIG. 17 and FIG. 18); this shows that these biomarkers are responsive to both positive and negative mood states changes.

A subset of proteins with altered abundance in the Test group are membrane-bound, an appealing characteristic as these potential biomarkers could possibly be identified without requiring exosome lysis in future studies.

In a single Test group participant, 4 of the proteins (BPIFA2, CSTB, PIGR and PKM) with altered abundance identified from pan-exosome samples were qualitatively validated using global proteomics/Proteome Discoverer in neuron-derived exosomes. A notable increase in fold-change was observed when using a targeted mass spectrometry approach for the neuron-derived exosomes. This discrepancy most likely results from a systematic underestimation of quantitative ratios caused by co-fragmentation of undesirable peptides when using isobaric mass tags such as those used in the untargeted proteomic analysis. This ratio compression does not occur when using targeted, label-free quantification strategies resulting in more pronounced fold changes. Taking this into account, smaller fold changes need to be considered significant when using isobaric mass tags for quantitative proteomics in the discovery group analysis and proteins of interest should be further analyzed using targeted approaches. Alternatively, these augmented fold-change results may also be due to enhanced protein responses in neuron-derived exosomes which become reduced in magnitude when diluted in total exosomes.

miRNA analysis on the Test group using the NanoString platform identified 34 miRNAs to be significantly changed in between pre- and post-work shifts. These measurements were subsequently validated for 2 miRNAs (miR1296-3p and miR519d-3p) using qPCR, providing reassurance of the reliability of the NanoString platform. Several of these miRNAs were also found to exhibit changes in abundance opposite to that of identified protein encoded by their target genes, suggesting a mechanism of gene regulation that is influencing the abundances of identified proteins. Omics analyses in the Test group confirmed the ability to identify exosomal proteins and their associated miRNAs that are detectable and may be altered pre- and post-work shift.

Global proteomic analysis of the larger Discovery group samples identified a considerably greater number of significantly changed proteins, including increases in AMY1A, BPI fold-containing family A member (BPIFA2), dipeptidyl peptidase 4 (DPP4), and decreases in small proline-rich protein 3 (SPRR3), fatty acid-binding protein 5 (FABP5), 14-3-3 protein zeta/delta (YWHAZ) and DMBT1. Four proteins that were altered-AMY1A (increased), CSTB (decreased), PIGR (decreased), and DMBT1 (decreased)—are known to be associated with CFS. Several other proteins, including liver-enriched gene 1 (LEG1), carbonic anhydrase 6 (CA6), suprabasin (SBSN), phosphoglycerate kinase 1 (PGK1) and cellular retinoic acid-binding protein 5, demonstrated close to significant changes in abundance and were also considered potential biomarkers. Gene set enrichment, GO classification and pathway analysis and functional protein network analysis were utilized to help understand the potential biological roles of the identified proteins In the Discovery group that reported increased mood disturbance and fatigue as the result of a long work shift. These analyses highlighted the potential existence of regulated fatigue-associated protein networks that generate ATP in response energy demand or cellular stress. PGK1 is an enzyme that catalyzes the formation of ATP from ADP and 1,3-diphosphoglycerate, playing an important role in glycolysis and ATP production. AMY1A hydrolyzes 1,4-alpha-glucoside bonds in oligosaccharides and polysaccharides, yielding glucose that can be used to generate ATP. DPP4 also influences glucose levels by deactivating incretins, which normally stimulate the release of insulin from the pancreas.

NanoString miRNA analysis on the Discovery group identified 73 miRNAs to be significantly changed and correlated with PoMS TMD or FI. Interestingly, some of these miRNAs have been reported to exhibit changes in other CNS pathologies. For example, hsa-miR-142-3p has been shown to be increased in individuals who have experienced a mild traumatic brain injury. It was hypothesize that the expression of these miRNAs are sensitive to changes in cognitive function and that they regulate the expression of biologically relevant proteins and pathways. Integrated analysis of the two-omics datasets was used to determine if any significantly altered miRNAs were known to regulate target genes encoding significantly altered proteins. miRNA-protein/gene pairs were selected if direction of change in the miRNA was in the opposite direction of the change in the protein, considering the typical mechanism of downregulation of a gene mRNA by upregulated miRNA. This analysis identified 3 miRNA-protein/gene pairs: miR-3185-PGK1, miR-642a-PIGR, and miR-134-YWHAZ.

Validation group saliva was used to determine if the candidate biomarkers identified in the Discovery group analysis correlated with TMD or FI. Proteomic analysis identified 12 of the 14 proteins altered in the Discovery group to be present in Validation group exosomes and, while not statistically significant, the mean fold-change of CSTB, DDP4, FABP5, PIGR and YWAZ maintained an inverse relationship with changes in TMD score. Additionally, PGK1 maintained and positive but weak correlation with changes in FI score. It should be noted that the magnitude of TMD and FI-difference scores in the validation group was significantly smaller than those of the Discovery group, which may make biomarker validation more challenging and is most likely evidenced by a lack of statistical significance. When the magnitude of FI score differences is considered, PGK1 would interesting protein for continued evaluation as a biomarker of cognitive fatigue. When the Validation group and Discovery group data is combined, the abundance of the other 6 candidate biomarkers (DPP4, BPIFA2, CA6, AMY1A, LEG1, and DMBT1) were still significantly altered between pre- and post-work saliva but were not determined to be associated with either TMD or FI. This notable observation highlights that many of the originally identified potential biomarkers may be associated biological processes altered by work alone that are not impacted by changes TMD or FI score. Therefore, these proteins may still have value as biomarkers of biologically relevant phenomenon unrelated to fatigue-associated cognitive impairment.

Of 13 miRNAs selected for continued evaluation in Validation group samples, the abundance miR3185 was correlated with TMD and FI. miR3185 was of particular interest because it is known to regulate target gene PGK1, a protein identified as a potential biomarker correlated with fatigue. The relation between miR3185 and PGK1 was strengthened by their inverse correlation, suggesting a potential biological mechanism for regulation of the PGK1 gene by miR3185 may be induced by fatigue. The inversely correlated levels of miR3185 and PGK1 could represent a coregulated set that are not only biomarkers of fatigue but could possibly contribute to a mechanism of fatigue induction or relief. The levels of miR3185 and PGK1 not only correlated with the degree of mood disturbance assessed by the POMS FI subscale, but the correlation extended beyond increased FI to decreased FI: among subjects whose PoMS FI difference were negative (reduced fatigue), miR3185 increased and PGK1 decreased.

PGK1 deficiency is associated with anemia syndromes that include progressive onset of weakness, fatigue, and lassitude and motor neuron vulnerability in spinal muscular atrophy (SMA). The increase in PGK1 with increased mood disturbance that was observed may represent a compensatory response to boost energy levels; but this is speculative and might be elucidated by following PGK1 levels in salivary exosomes over time during a demanding work shift.

Little is known about miR3185, other than it is specific to primate genomes, and reported to be increased in cardiac tissues in cases of mechanical asphyxia as well as associated with increased survival in liver cancer. PGK1 and miR3185 are both attractive biomarker targets that could potentially be used to detect the onset of fatigue-associated cognitive impairment in salivary exosomes.

In conclusion, the results herein identify proteins and miRNAs that correlate to changes in mood states, including FI, as measured by the PoMS questionnaire. They represent possible biomarkers that can be quantified using salivary exosomes with the potential to reveal an increased risk for decline in cognitive performance. These results add to the growing knowledge of detectable changes in the biomolecular composition of exosomes in various pathologies and point to a promising candidate biomarker, PGK1, in saliva as well as suggest a possible mechanism in which expression of the PGK1 gene is regulated by miR3185 in response to changes in fatigue. This biomarker requires further validation in larger well-defined cohorts. The limitations of the current study were small sample size and potential inaccuracies associated with subjective self-assessment of mood states. Despite these limitations, this study demonstrates the value of using an integrated multi-omics approach for the identification of novel disease-associated mechanisms and biomarker in salivary exosomes and could be used to develop a rapid saliva-based test for cognitive fatigue.

Table 1 shows additional proteins that change with cognitive fatigue and Table 2 shows additional microRNAs that change with cognitive fatigue.

TABLE 1

| | | | Fold |
|---|---|---|---|
| | | Fold | Change |
| Gene | Protein Description | Change | P-Value |
| LEG1 | Liver-enriched gene 1 protein | 4.46 | 0.079 |
| AMY1A | Alpha-amylase 1 | 1.85 | 0.002 |
| BPIFA2 | BPI fold-containing family A member | 1.64 | 0.006 |
| CA6 | Carbonic anhydrase 6 | 1.50 | 0.083 |
| DPP4 | Dipeptidyl peptidase 4 | 1.24 | 0.030 |
| SBSN | Suprabasin | −1.23 | 0.060 |
| PIGR | Polymeric immunoglobulin receptor | −1.28 | 0.063 |
| CSTB | Cystatin-B | −1.34 | 0.057 |
| SPRR3 | Small proline-rich protein 3 | −1.49 | 0.010 |
| CRABP2 | Cellular retinoic acid-binding protein 2 | −1.49 | 0.098 |
| FABP5 | Fatty acid-binding protein 5 | −1.72 | 0.004 |
| YWHAZ | 14-3-3 protein zeta/delta | −2.22 | 0.036 |
| DMBT1 | Deleted in malignant brain tumors 1 | −2.28 | 0.015 |

TABLE 2

| | Accession | Fold | |
|---|---|---|---|
| miR | Number | Change^{a} | p-value |
| hsa-miR-1-5p | MIMAT0031892 | 1.49 | 0.07 |
| hsa-miR-18b-5p | MIMAT0001412 | 1.29 | 0.01 |
| hsa-miR-24-3p | MIMAT0000080 | 1.20 | 0.04 |
| hsa-miR-29a-3p | MIMAT0000086 | 1.08 | 0.01 |
| hsa-miR-30e-5p | MIMAT0000692 | 1.46 | 0.01 |
| hsa-miR-96-5p | MIMAT0000095 | 1.28 | 0.07 |
| hsa-miR-98-5p | MIMAT0000096 | 1.87 | 0.01 |
| hsa-miR-105-5p | MIMAT0000102 | 1.79 | 0.01 |
| hsa-miR-126-3p | MIMAT0000445 | 1.40 | 0.07 |
| hsa-miR-126-3p | MIMAT0000445 | 1.40 | 0.07 |
| hsa-miR-128-2-5p | MIMAT0031095 | 1.33 | 0.04 |
| hsa-miR-134-3p | MIMAT0000447 | 1.50 | 0.06 |
| hsa-miR-140-5p | MIMAT0000431 | 2.01 | 0.00 |
| hsa-miR-142-3p | MIMAT0000434 | 1.55 | 0.04 |
| hsa-miR-181a-2-3p | MIMAT0004558 | −1.31 | 0.03 |
| hsa-miR-181b-5p + | MIMAT0000257 | −1.25 | 0.02 |
| hsa-miR-181d-5p^{b} | MIMAT0002821 | | |
| hsa-miR-182-5p | MIMAT0000259 | −1.77 | 0.03 |
| hsa-miR-200c-3p | MIMAT0000617 | 1.51 | 0.04 |
| hsa-miR-203a-3p | MIMAT0000264 | 1.30 | 0.04 |
| hsa-miR-210-3p | MIMAT0000267 | −1.21 | 0.03 |
| hsa-miR-214-3p | MIMAT0000271 | 1.53 | 0.01 |
| hsa-miR-219a-2-3p | MIMAT0000271 | −1.35 | 0.01 |
| hsa-miR-221-3p | MIMAT0000278 | −1.52 | 0.01 |
| hsa-miR-329-3p | MIMAT0001629 | −1.28 | 0.01 |
| hsa-miR-339-5p | MIMAT0004702 | 1.34 | 0.02 |
| hsa-miR-371a-5p | MIMAT0004687 | −1.43 | 0.03 |
| hsa-miR-374b-5p | MIMAT0004955 | 1.29 | 0.05 |
| hsa-miR-376b-3p | MIMAT0002172 | 1.43 | 0.01 |
| hsa-miR-380-3p | MIMAT0000735 | 1.64 | 0.05 |
| hsa-miR-381-3p | MIMAT0000736 | 1.30 | 0.01 |
| hsa-miR-410-3p | MIMAT0002171 | 1.59 | 0.04 |
| hsa-miR-485-5p | MIMAT0002175 | 1.74 | 0.01 |
| hsa-miR-497-5p | MIMAT0002820 | 0.77 | 0.04 |
| hsa-miR-503-3p | MIMAT0022925 | 1.32 | 0.02 |
| hsa-miR-509-3-5p | MIMAT0004975 | 1.36 | 0.01 |
| hsa-miR-511-5p | MIMAT0002808 | −1.48 | 0.03 |
| hsa-miR-513b-5p | MIMAT0005788 | −2.27 | 0.00 |
| hsa-miR-513c-5p | MIMAT0005789 | 1.58 | 0.03 |
| hsa-miR-518e-3p | MIMAT0002861 | −1.93 | 0.03 |
| hsa-miR-519d-3p | MIMAT0002853 | 1.43 | 0.04 |
| hsa-miR-532-3p | MIMAT0004780 | 1.27 | 0.02 |
| hsa-miR-548e-3p | MIMAT0005874 | 1.50 | 0.03 |
| hsa-miR-598-3p | MIMAT0003266 | −1.33 | 0.03 |
| hsa-miR-614 | MIMAT0003282 | −1.71 | 0.01 |
| hsa-miR-619-3p | MIMAT0003288 | 2.15 | 0.01 |
| hsa-miR-642a-5p | MIMAT0020924 | 1.90 | 0.00 |
| hsa-miR-648 | MIMAT0003318 | −1.91 | 0.04 |
| hsa-miR-654-3p | MIMAT0004814 | 1.57 | 0.01 |
| hsa-miR-655-3p | MIMAT0003331 | −1.46 | 0.03 |
| hsa-miR-660-3p | MIMAT0022711 | 1.31 | 0.01 |
| hsa-miR-663a | MIMAT0003326 | −1.50 | 0.03 |
| hsa-miR-664b-5p | MIMAT0022271 | 2.10 | 0.10 |
| hsa-miR-765 | MIMAT0003945 | −1.28 | 0.05 |
| hsa-miR-873-3p | MIMAT0022717 | 1.24 | 0.03 |
| hsa-miR-1245a | MIMAT0005897 | 1.60 | 0.01 |
| hsa-miR-1248 | MIMAT0005900 | 1.56 | 0.06 |
| hsa-miR-1255a | MIMAT0005906 | −1.28 | 0.01 |
| hsa-miR-1257 | MIMAT0005908 | 1.45 | 0.00 |
| hsa-miR-1269a | MIMAT0026637 | 1.27 | 0.10 |
| hsa-miR-1270 | MIMAT0005924 | 1.34 | 0.03 |
| hsa-miR-1289 | MIMAT0005879 | 1.32 | 0.04 |
| hsa-miR-1296-3p | MIMAT0026637 | 1.22 | 0.03 |
| hsa-miR-1304-3p | MIMAT0022720 | 1.88 | 0.02 |
| hsa-miR-1910-3p | MIMAT0026917 | −1.32 | 0.04 |
| hsa-miR-2110 | MIMAT0010133 | −1.44 | 0.03 |
| hsa-miR-3131 | MIMAT0014996 | 1.47 | 0.02 |
| hsa-miR-3140-5p | MIMAT0019204 | 1.75 | 0.05 |
| hsa-miR-3147 | MIMAT0015019 | 1.47 | 0.04 |
| hsa-miR-4454 + | MIMAT0018976 | −1.64 | 0.01 |
| hsa-miR-7975^{b} | MIMAT0031178 | | |
| hsa-miR-4536-5p | MIMAT0019078 | 1.85 | 0.01 |
| hsa-miR-525-3p | MIMAT0002839 | 1.94 | 0.003 |
| hsa-miR-545-3p | MIMAT0003165 | 1.81 | 0.032 |
| hsa-miR-1252-5p | MIMAT0005944 | 1.77 | 0.01 |
| hsa-miR-376a-2-5p | MIMAT0022928 | 1.42 | 0.047 |
| hsa-miR-3690 | MIMAT0018119 | 1.34 | 0.014 |
| hsa-miR-4536-3p | MIMAT0020959 | 1.28 | 0.017 |
| hsa-miR-551b-3p | MIMAT0003233 | 1.24 | 0.044 |
| hsa-let-7e-5p | MIMAT0000066 | 1.23 | 0.024 |
| hsa-miR-196a-5p | MIMAT0000226 | −1.22 | 0.038 |
| hsa-miR-203a-5p | MIMAT0031890 | −1.23 | 0.001 |
| hsa-let-7i-5p | MIMAT0000415 | −1.26 | 0.021 |
| hsa-miR-21-5p | MIMAT0000076 | −1.27 | 0.037 |
| hsa-miR-626 | MIMAT0003295 | −1.28 | 0.021 |
| hsa-let-7f-5p | MIMAT0000067 | −1.39 | 0.028 |
| hsa-miR-885-3p | MIMAT0004948 | −1.39 | 0.043 |
| hsa-let-7b-5p | MIMAT0000063 | −1.40 | 0.017 |
| hsa-miR-487b-3p | MIMAT0003180 | −1.43 | 0.023 |
| hsa-miR-548j-5p | MIMAT0005875 | −1.45 | 0.044 |
| hsa-miR-221-3p | MIMAT0000278 | −1.53 | 0.012 |
| hsa-miR-555 | MIMAT0003219 | −1.54 | 0.041 |
| hsa-miR-152-5p | MIMAT0026479 | −1.57 | 0.015 |
| hsa-miR-1204 | MIMAT0005868 | −1.86 | 0.015 |

^{a}As compared to normal controls
^{b}The presence of one miR over the other could not be distinguished because of assay limitations. Thus “+” indicates one or the other or both miRs.

As used herein, a “cognitive fatigue” refers to refers to a decrease in cognitive function resulting from sustained demands on cognitive function over a period of time, independent of disease, infection, brain injury, and genetic and physical abnormalities. That is, for cognitive fatigue, the decrease in cognitive function is measured from the given subject's cognitive function prior to being subjected to a period of sustained demands on cognitive function or as compared to the average cognitive function of a population of similarly situated individuals prior to being subjected to a period of sustained demands on cognitive function. Thus, the “normal control” used for comparison in determining whether a subject suffers from cognitive fatigue is the given subject's state prior to the period of sustained cognitive function or the average state of a population of similarly situated individuals prior being subjected to a period of sustained cognitive function.

In some embodiments, the period of sustained cognitive function may be intermittently interrupted by one or more breaks, the time of each break independently being about 1 to about 45 minutes, with the ratio of the sum amount of the one or more breaks to the given period of sustained cognitive function being about 1:8 or less, about 1:9 or less, about 1:10 or less, about 1:11 or less, about 1:12 or less, about 1:13 or less, about 1:14 or less, about 1:15 or less, about 1:16 or less, about 1:17 or less, about 1:18 or less, about 1:19 or less, about 1:20 or less, about 1:21 or less, about 1:22 or less, about 1:23 or less, about 1:24 or less, or about 1:25 or less.

Subjects suffering from cognitive fatigue may also independently suffer from another cognitive impairment. For example, a person suffering from Alzheimer's disease or genetic chronic fatigue syndrome may also suffer from cognitive fatigue after being subjected to a sustained demand of cognitive function for a given period of time.

As used herein, “cognitive function” refers to the deliberate and conscious performance of some cognitive activity, such as memory, perception, learning, and reasoning. “Learning” refers to acquisition of information and/or knowledge, and is typically evaluated by exposing a subject to a learning experience and observing changes in behavior arising from that experience. “Memory” refers to the storage and retrieval of information. Memory is generally classified into short term memory (also called working memory) and long-term memory, where consolidation into long term memory is believed occur through a stage involving short term memory.

Exemplary stimulants include caffeine, Huperzine-A, L-theanine, amphetamine, dextroamphetamine, methylphenidate, dexmethylphenidate, atomoxetine, lisdexamfetamine, and the like.

In some embodiments, the present invention is directed the use of PGK1 and/or miR3185 for the detection of cognitive fatigue in subjects. As provided herein, “PGK1” refers to a protein having at least 95%, at least 96%, at least 97%, at least 98%, at least 99%, or 100% sequence identity to human phosphoglycerate kinase 1 (Accession No. NP_000282.1). As provided herein, “miR3185” refers to an RNA sequence having no more than 3 base differences compared to hsa-miR-3185 (Accession No. MIMAT0015065).

Proteins, antibodies, and microRNAs may be made using methods in the art including chemical synthesis, biosynthesis or in vitro synthesis using recombinant DNA methods, and solid phase synthesis and purified using methods in the art such as reverse phase high-performance liquid chromatography (HPLC), ion-exchange or immunoaffinity chromatography, filtration or size exclusion, or electrophoresis.

As used herein, an “isolated” compound refers to a compound that is isolated from its native environment. For example, an isolated polynucleotide is a one which does not have the bases normally flanking 5′ end and/or 3′ end of the polynucleotide as it is found in nature. As another example, an isolated polypeptide is a one which does not have its native amino acids, which correspond to the full-length polypeptide, flanking the N-terminus, C-terminus, or both.

As used herein, “antibody” refers to naturally occurring and synthetic immunoglobulin molecules and immunologically active portions thereof (i.e., molecules that contain an antigen binding site that specifically bind the molecule to which antibody is directed against). As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include: single chain Fvs (scFvs), Fab fragments, Fab′ fragments, F(ab′)₂, disulfide linked Fvs (sdFvs), Fvs, and fragments comprising or alternatively consisting of, either a VL or a VH domain.

As used herein, a compound (e.g., receptor or antibody) “specifically binds” a given target (e.g., ligand or epitope) if it reacts or associates more frequently, more rapidly, with greater duration, and/or with greater binding affinity with the given target than it does with a given alternative, and/or indiscriminate binding that gives rise to non-specific binding and/or background binding. As used herein, “non-specific binding” and “background binding” refer to an interaction that is not dependent on the presence of a specific structure (e.g., a given epitope). An example of an antibody that specifically binds PGK1 is an antibody that binds PGK1 with greater affinity, avidity, more readily, and/or with greater duration than it does to other compounds. An antibody that specifically binds PGK1 over a specified alternative is an antibody that binds PGK1 with greater affinity, avidity, more readily, and/or with greater duration than it does to the specified alternative. An antibody that specifically binds a given epitope of PGK1 is an antibody that binds the given epitope with greater affinity, avidity, more readily, and/or with greater duration than it does to other epitopes of PGK1. As used herein, an “epitope” is the part of a molecule that is recognized by an antibody. Epitopes may be linear epitopes or three-dimensional epitopes. As used herein, the terms “linear epitope” and “sequential epitope” are used interchangeably to refer to a primary structure of an antigen, e.g., a linear sequence of consecutive amino acid residues, that is recognized by an antibody. As used herein, the terms “three-dimensional epitope” and “conformational epitope” are used interchangeably to refer a three-dimensional structure that is recognized by an antibody, e.g., a plurality of non-linear amino acid residues that together form an epitope when a protein is folded.

As used herein, “binding affinity” refers to the propensity of a compound to associate with (or alternatively dissociate from) a given target and may be expressed in terms of its dissociation constant, Kd. In some embodiments, the antibodies have a Kd of 10-5 or less, 10-6 or less, preferably 10-7 or less, more preferably 10-8 or less, even more preferably 10-9 or less, and most preferably 10-10 or less, to their given target. Binding affinity can be determined using methods in the art, such as equilibrium dialysis, equilibrium binding, gel filtration, immunoassays, surface plasmon resonance, and spectroscopy using experimental conditions that exemplify the conditions under which the compound and the given target may come into contact and/or interact. Dissociation constants may be used determine the binding affinity of a compound for a given target relative to a specified alternative. Alternatively, methods in the art, e.g., immunoassays, in vivo or in vitro assays for functional activity, etc., may be used to determine the binding affinity of the compound for the given target relative to the specified alternative. Thus, in some embodiments, the binding affinity of the antibody for the given target is at least 1-fold or more, preferably at least 5-fold or more, more preferably at least 10-fold or more, and most preferably at least 100-fold or more than its binding affinity for the specified alternative.

As used herein, the term “sample” is used in its broadest sense and includes specimens and cultures obtained from any source, as well as biological samples and environmental samples. Biological samples may be obtained from animals (including humans) and encompass fluids, solids, tissues, and gases. Biological samples include blood products, such as plasma, serum, and the like. A biological sample can be obtained from a subject using methods in the art. A sample to be analyzed using one or more methods described herein can be either an initial unprocessed sample taken from a subject or a subsequently processed, e.g., partially purified, diluted, concentrated, fluidized, pretreated with a reagent (e.g., protease inhibitor, anti-coagulant, etc.), and the like. In some embodiments, the sample is a blood sample. In some embodiments, the blood sample is a whole blood sample, a serum sample, or a plasma sample. In some embodiments, the sample may be processed, e.g., condensed, diluted, partially purified, and the like. In some embodiments, the sample is pretreated with a reagent, e.g., a protease inhibitor. In some embodiments, two or more samples are collected at different time intervals to assess any difference in the amount of the analyte of interest, the progression of a disease or disorder, or the efficacy of a treatment. The test sample is then contacted with a capture reagent and, if the analyte is present, a conjugate between the analyte and the capture reagent is formed and is detected and/or measured with a detection reagent. In some embodiments, the sample is saliva. In some embodiments, the sample is one or more exosomes obtained from saliva.

As used herein, a “capture reagent” refers to a molecule which specifically binds an analyte of interest. The capture reagent may be immobilized on a assay substrate. For example, if the analyte of interest is an antibody, the capture reagent may be an antigen or an epitope thereof to which the antibody specifically binds.

As used herein, an “assay substrate” refers to any substrate that may be used to immobilize a capture reagent thereon and then detect an analyte when bound thereto. Examples of assay substrates include membranes, beads, slides, and multi-well plates.

As used herein, a “detection reagent” refers to a substance that has a detectable label attached thereto and specifically binds an analyte of interest or a conjugate of the analyte of interest, e.g., an antibody-analyte conjugate.

As used herein, a “detectable label” is a compound or composition that produces or can be induced to produce a signal that is detectable by, e.g., visual, spectroscopic, photochemical, biochemical, immunochemical, or chemical means. The use of the term “labeled” as a modifier of a given substance, e.g., a labeled antibody, means that the substance has a detectable label attached thereto. A detectable label can be attached directly or indirectly by way of a linker (e.g., an amino acid linker or a chemical moiety). Examples of detectable labels include radioactive and non-radioactive isotopes (e.g., 125I, 18F, 13C, etc.), enzymes (e.g., β-galactosidase, peroxidase, etc.) and fragments thereof, enzyme substrates, enzyme inhibitors, coenzymes, catalysts, fluorophores (e.g., rhodamine, fluorescein isothiocyanate, etc.), dyes, chemiluminescers and luminescers (e.g., dioxetanes, luciferin, etc.), and sensitizers. A substance, e.g., antibody, having a detectable label means that a detectable label that is not linked, conjugated, or covalently attached to the substance, in its naturally-occurring form, has been linked, conjugated, or covalently attached to the substance by the hand of man. As used herein, the phrase “by the hand of man” means that a person or an object under the direction of a person (e.g., a robot or a machine operated or programmed by a person), not nature itself, has performed the specified act. Thus, the steps set forth in the claims are performed by the hand of man, e.g., a person or an object under the direction of the person.

### Immunoassays

In some embodiments, the present invention provides immunoassays for detecting PGK1 in a sample, e.g., a biological sample, obtained from a subject. Such assays include any immunoassay format in the art such as enzyme immune assays (EIAs), magnetic immunoassays (MIAs), counting immunoassays (CIAs), chemiluminescent immunoassays (CLIAs), radioimmunoassays (RIAs), electrochemiluminescence immunoassays (ECLIA), fluorescent immunoassays (FIA), enzyme-linked immunosorbent assays (ELISAs), Western blot assays, and lateral flow tests (LFTs), and the like. The assays may be automated or manual. The various assays may employ any suitable labeling and detection system. The sensitivity and specificity of the assays can be further improved by optimizing the assay conditions, e.g., reaction times and temperatures, and/or modifying or substituting the reagents, e.g., different detection and labeling system, using methods in the art. In some embodiments, the immunoassay is an ELISA assay. In some embodiments, the immunoassay is a sandwich ELISA assay. In some embodiments, the immunoassay is a lateral flow assay.

In some embodiments, the sample to be tested is concentrated and then the level of PGK1 is measured in the concentrated sample and the level of PGK1 in the unconcentrated sample is mathematically extrapolated from the degree of concentration.

### Kits

In some embodiments, the present invention provides kits for assaying PGK1 and/or miR3185 in a sample, e.g., a biological sample, obtained from a subject. In some embodiments, the kits comprise a capture reagent that specifically binds the PGK1 and/or miR3185 packaged together with a detection reagent for detecting and/or measuring any PGK1 and/or miR3185 conjugated with the capture reagent. In some embodiments, the kits comprise an assay substrate for performing an immunoassay and immobilizing the capture reagent thereto. In some embodiments, the assay substrate is a lateral flow test (LFT) test strip that has immobilized thereon a capture reagent for PGK1 and/or a capture reagent for miR3185. In some embodiments, the kits comprise one or more reagents, e.g., blocking buffers, assay buffers, diluents, wash solutions, etc., for assaying the target analyte. In some embodiments, the kits comprise additional components such as interpretive information, control samples, reference levels, and standards. In some embodiments, the kits further comprise one or more therapeutic agents, e.g., a stimulant, for preventing, inhibiting, reducing, or treating cognitive fatigue in a subject.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

### Diagnostic and Prognostic Applications

The methods and kits as contemplated herein may be used in the evaluation of a cognitive fatigue. The methods and kits may be used to monitor the progress of such a disease, assess the efficacy of a treatment for the disease, and/or identify patients suitable for a given treatment in a subject. The methods and kits may be used to diagnose a subject as having a cognitive fatigue and/or provide the subject with a prognosis.

In some embodiments, the methods and kits may be used to determine whether a subject exhibits a level of PGK1 and/or miR3185 is low or high as compared to a control. In some embodiments, the control is a sample is obtained from the given subject prior to a period of sustained cognitive function or a pooled sample of samples obtained from a population of similarly situated individuals prior to being subjected to a period of sustained cognitive function. In some embodiments, the control is a given reference level based on a sample is obtained from the given subject prior to a period of sustained cognitive function or a pooled sample of samples obtained from a population of similarly situated individuals prior to being subjected to a period of sustained cognitive function. In some embodiments, the given reference level is a baseline level that was obtained from the subject when the subject was well-rested. The change in the level of PGK1 and/or miR3185 may then be used to determine whether the subject is suffering from a cognitive fatigue.

A subject identified as suffering from cognitive fatigue may be subjected to a suitable treatment, e.g., a period of rest from activities that are demanding of cognitive function or a stimulant.

### Non-Clinical Applications

In some embodiments, the methods and kits may be used for research purposes. For example, the methods and kits may be used to identify activities that are more demanding of cognitive function than other activities by, e.g., measuring the amount of change in one or more biomarkers resulting from a first activity and comparing to the amount of change of the same biomarkers resulting from a second activity. In some embodiments, the methods and kits may be used to study mechanisms involved in cognitive fatigue. In some embodiments, the methods and kits may be used to develop and screen for therapeutics that inhibit, reduce, or alleviate cognitive fatigue.

The following examples are intended to illustrate but not to limit the invention.

### EXAMPLES

### Participants

Saliva donors were recruited from UCLA medical and dental residents. A total of 36 residents participated. The research was approved by the UCLA IRB committee (UCLA IRB #17-000317). Residents were given information about the research, and they gave oral consent for participation in this study.

### Whole Saliva Collection

Saliva samples were collected from 36 medical and dental residents before and after their 12-hour work shift. Participates were given a 50 mL conical tube to collect saliva sample for maximum 1 hour duration. Saliva samples were centrifuged at 2600 rcf at 4° C. for 15 minutes. Supernatant was aliquoted to 1 mL each vial. For each vial, 1 μL of Superase RNase inhibitor (ThermoFisher #AM2694) was added. Saliva samples are stored at −80° C. until processing.

### The Profile of Mood States (PoMS) Questionnaire

Mood states were accessed using a modified version of the PoMS questionnaire (McNair et al., 1971 & 1992; Heuchert & McNair, 2012; Lin et al., 2014; Albrecht et al., 1989). The modified questionnaire consists of a 62-item inventory of six subscales: tension-anxiety (TA), depression-dejection (DD), anger-hostility (AH), vigor-activity (VA), fatigue-inertia (FI) and confusion-bewilderment (CB). Responses were provided on a 5-point scale range from 1 (Not at all) to 5 (extremely). The global indicator Total Mood Disturbance (TMD) is defined as: TMD=(AH+CB+DD+FI+TA)−VA. An increase in TMD suggests the onset of mood disturbances that would be considered unfavorable for optimum cognitive performance, such as increased fatigue; decreases in TMD reflect positive changes in mood, for example, a decrease in tension and anxiety.

### PoMS Statistical Analysis

Complete PoMS data with both pre- and post-shift scores from 36 residents were included in analysis. Wilcoxon signed rank test was used for the paired comparison between pre and post work shift. The analysis was performed using SAS version 9.4 (SAS Institute Inc., Cary, NC).

### Separation of Saliva Samples by PoMS TMD Score

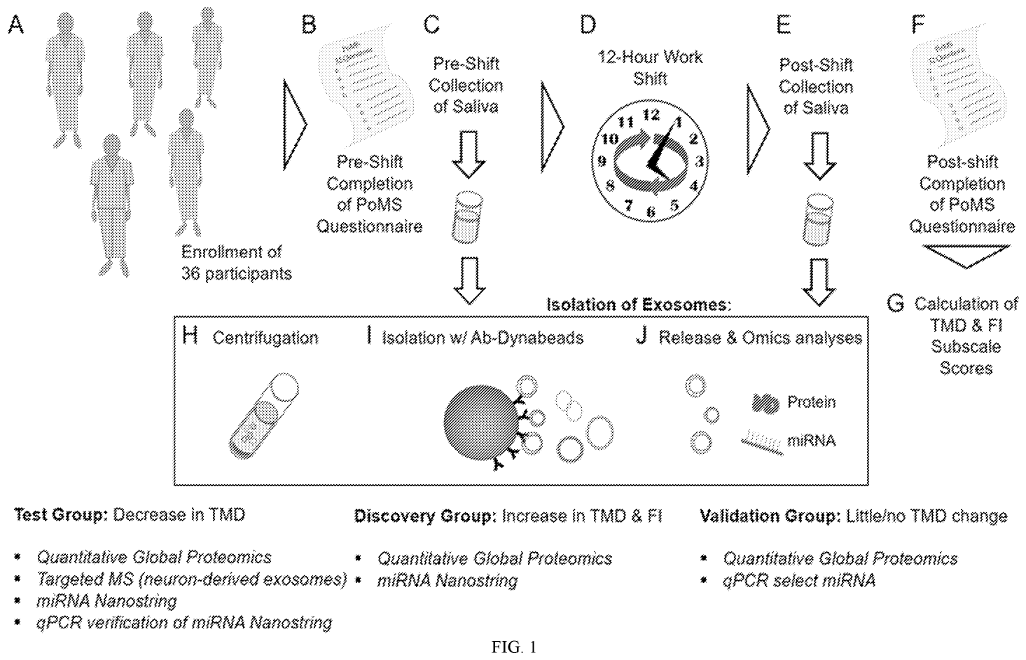

Based on TMD, subject saliva samples were assigned to the Test group, Discovery group or Validation group and the samples were assessed using different methods (FIG. 1). The Test group comprised saliva from 6 participants with a negative TMD difference, that is, those who reported no change or an improvement in mood as a result of the work shift. Samples from these participants were used to optimize the isolation of exosomes and Omic analysis methods. The Discovery group comprised 20 subjects with the greatest increase in TMD score post-shift; their saliva samples underwent proteomics and Nanostring miRNA analyses. The Validation group consisted of 10 subjects with nearly unchanged or only slightly increased TMD post-shift scores and their saliva samples were used to validate biomarkers and their directional changes identified from the Discovery group. For the Validation group, targeted proteomics was used for select proteins and qPCR of select genes rather than Nanostring analysis.

### EV Isolation and Enrichment

Saliva EVs were isolated using magnetic microsphere-based immunoprecipitation (IP) modified from established methods (Heinzelman et al., 2019). Frozen saliva aliquots were quickly thawed at 37° C., spiked with HALT Protease and Phosphatase Inhibitor Cocktail (Thermo Fisher Scientific Cat #78440), diluted 3-fold with ice cold 1×PBS and centrifuged (13,000 rcf, 20 min, 4° C.) to remove debris. Supernatants were then incubated (overnight, 4° C.) with a mixture of antibodies specific for various exosomal surface markers including tetraspanins CD9, CD63, CD81 (all Thermo Fisher Scientific, 10626D, 10628D, and 10630D, respectively) that were previously desalted (Zeba™ Spin Desalting Columns, 7K MWCO, 0.5 mL, Thermo Fisher Scientific Cat #89882) and conjugated to Dynabeads (Invitrogen DYNAL Dynabeads M-270 Epoxy, Thermo Fisher Scientific Cat #14301) according to the manufacturer's protocols. The isolated exosomes were used for proteomics by mass spectroscopy (MS) and miRNA analysis.

For isolation of salivary exosomes originating from neurons (performed for a single participant in the Test Group), Dynabeads conjugated to antibodies specific for a neuronal surface marker CD171 (Thermo Fisher Scientific, MA5-14140) was used. After incubation, the beads destined for proteomics analysis were subsequently washed once with 1×PBS, twice with 0.15 M citrate phosphate buffer (pH 5.2), and once again with 1×PBS. For beads destined for miRNA analysis, 0.1% BSA was added to both 1×PBS washes. The isolated exosomes were used for targeted proteomics by MS.

### Quantitative Global Proteomics Analysis

Immunoprecipitated exosomes were eluted from the Dynabeads by heating (95° C., 5 min) in lysis buffer (100 μL, 12 mM sodium lauroyl sarcosine, 0.5% sodium deoxycholate, 50 mM triethylammonium bicarbonate (TEAB), Halt™ Protease and Phosphatase Inhibitor Cocktail) then subjected to bath sonication (Bioruptor Pico, Diagenode Inc.; Denville, NJ) for 10 min. The samples were treated with tris (2-carboxyethyl) phosphine (10 μL, 55 mM in 50 mM TEAB, 30 min, 37° C.) followed by treatment with chloroacetamide (10 μL, 120 mM in 50 mM TEAB, 30 min, 25° C. in the dark). They were then diluted 5-fold with aqueous 50 mM TEAB and incubated overnight with Sequencing Grade Modified Trypsin (1 μg in 10 μL of 50 mM TEAB; Promega Cat #V511A, Madison, WI). Following this an equal volume of ethyl acetate/trifluoroacetic acid (TFA, 100/1, v/v) was added and after vigorous mixing (5 min) and centrifugation (13,000×g, 5 min), the supernatants were discarded, and the lower phases were dried in a centrifugal vacuum concentrator. The samples were then desalted using a modified version of Rappsilber's protocol (Rappsilber et al., 2017) in which the dried samples were reconstituted in acetonitrile/water/TFA (solvent A, 100 μL, 2/98/0.1, v/v/v) and then loaded onto a small portion of a C18-silica disk (3M, Maplewood, MN) placed in a 200 μL pipette tip. Prior to sample loading the C18 disk was prepared by sequential treatment with methanol (20 μL), acetonitrile/water/TFA (solvent B, 20 μL, 80/20/0.1, v/v/v) and finally with solvent A (20 μL). After loading the sample, the disc was washed with solvent A (20 μL, eluent discarded) and eluted with solvent B (40 μL). The collected eluent was dried in a centrifugal vacuum concentrator. The samples were then chemically modified using a TMT11plex Isobaric Label Reagent Set (Thermo Fisher Scientific) as per the manufacturer's protocol. The TMT-labeled peptides were dried and reconstituted in solvent A (50 μL), and an aliquot (2 μL) was taken for measurement of total peptide concentration (Pierce Quantitative Colorimetric Peptide, Thermo Fisher Scientific). The samples were then pooled and desalted using the modified Rappsilber's protocol. The eluants were then dried and reconstituted in water/acetonitrile/FA (solvent E, 10 μL, 98/2/0.1, v/v/v), and aliquots (5 μL) were injected onto a reverse phase nanobore HPLC column (AcuTech Scientific, C18, 1.8 μm particle size, 360 μm×20 cm, 150 μm ID), equilibrated in solvent E and eluted (500 nL/min) with an increasing concentration of solvent F (acetonitrile/water/FA, 98/2/0.1, v/v/v: min/% F; 0/0, 5/3, 18/7, 74/12, 144/24, 153/27, 162/40, 164/80, 174/80, 176/0, 180/0) using an Eksigent NanoLC-2D system (Sciex (Framingham, MA)). The effluent from the column was directed to a nanospray ionization source connected to a hybrid quadrupole-Orbitrap mass spectrometer (Q Exactive Plus, Thermo Fisher Scientific) acquiring mass spectra in a data-dependent mode alternating between a full scan (m/z 350-1700, automated gain control (AGC) target 3×106, 50 ms maximum injection time, FWHM resolution 70,000 at m/z 200) and up to 15 MS/MS scans (quadrupole isolation of charge states 2-7, isolation window 0.7 m/z) with previously optimized fragmentation conditions (normalized collision energy of 32, dynamic exclusion of 30 s, AGC target 1×105, 100 ms maximum injection time, FWHM resolution 35,000 at m/z 200).

### Protein Quantification and Statistical Analysis

Raw proteomic data were searched against the Uniprot human reviewed protein database using SEQUEST-HT in Proteome Discoverer (Version 2,4, Thermo Scientific), which provided measurements of relative abundance of the identified peptides. Decoy database searching was used to generate high confidence tryptic peptides (FDR<1%). Tryptic peptides containing amino acid sequences unique to individual proteins were used to identify and provide relative quantification between proteins in each sample. Between-group comparisons were analyzed using the abundance ratio p-value (Student's t-test).

### Protein Bioinformatic Analysis

Proteins exhibiting a fold change with a magnitude ≥1.2 and a p-value≤0.1 were subject to comprehensive gene set enrichment analysis gene ontology (GO) classification and KEGG (Kanehisa & Goto, 2000) pathway analysis using Enrichr (Chen et al., 2013), as well as functional protein association network analysis using the STRING database (version 11.5), which was used for functional interpretation of the proteomics data and provided p-values corrected by the FDR method (Szklarczyk et al., 2019).

### Targeted LC-MS/MS Protein Quantification

Proteins isolated by antibody-conjugated microbeads were reduced, alkylated, and treated with trypsin as described in Global Proteomics Analysis; however, in contrast with that sample processing protocol, no isotopically labeled chemical tags were utilized to provide relative quantification between peptides in different samples. Furthermore, the data were acquired with the mass spectrometer utilizing a customized targeted-selected ion monitoring/data-dependent MS/MS (t-SIM/dd-MS²) method in which an inclusion list was utilized to sample only select peptides corresponding to specific proteins were assessed using precursor ion peak areas. Data from the global proteomic analysis was used to identify unique peptides for this analysis and select the correct m/z (Da) and charge state (Z) of each peptide targeted. The sensitivity gained by the targeted analysis using the SIM scan (AGC target 2×10⁵, 130 ms maximum injection time, FWHM resolution 70,000 at m/z 200, isolation window 2.0 m/z) permitted modification of the LC gradient (min/% F; 0/0, 5/3, 55/22, 61/35, 63/80, 73/80, 75/0, 79/0) and shortening of mass spectrometer acquisition time.

Global miRNA Analysis

RNA was extracted from the immunoprecipitated saliva exosomes using the SeraMir Exosome RNA Column Purification Kit (System Biosciences (Palo Alto, CA)) according to the manufacturer's protocol. The quality of the RNA was assessed via RNA electrophoresis using the Small RNA Kit (Agilent Technologies, Santa Clara, CA) on a 2100 Bioanalyzer System (Agilent Technologies) according to the manufacturer's instructions. Global profiling of miRNA from Test and Discovery group samples was done at the UCLA Center for Systems Biomedicine with the nCounter Human v3 miRNA Expression Assay (NanoString Technologies; Seattle, WA), in which 800 pairs of probes specific for a predefined set of biologically relevant miRNAs were combined with a series of internal controls to form a Human miRNA Panel CodeSet (NanoString Technologies). MiRNA (100 ng) targets of interest were hybridized overnight with two juxta-positioned probes: a biotinylated capture probe and uniquely fluorescently labeled reporter probe for each target. The hybridized samples were then transferred to the nCounter Prep Station where excess probes were removed, and the target-probe complexes were immobilized and aligned on the surface of a flow cell using an automated liquid handler. The unique sequences of the reporter probes were counted using the nCounter Digital Analyzer and translated into the number of counts per miRNA target. nSolver Analysis Software (NanoString Technologies) was used to facilitate data extraction and analysis. A paired t-test revealed several miRNAs exhibiting significant changes in abundance in response to work shifts (fold change magnitude ≥1.2; p-value≤0.05).

### Targeted MiRNA Analysis

Quantitative polymerase chain reaction (qPCR) was used to verify the miRNA levels detected in the NanoString analysis. For verification, the same RNA samples that were used in the NanoString analysis were assayed for select miRNAs using the TaqMan Advanced miRNA Assay (Thermo Fisher Scientific, Cat #A25576) according to the manufacturer's protocol. qPCR amplification Ct values for each resident's samples were compared pre- and post-shift using the ΔΔCt method and converted to a % change in miRNA levels (post-work-pre-work) for each subject.

Identification of miRNA Target Genes

Target genes associated with miRNAs exhibiting significant changes in abundance in response to work shifts (fold change magnitude ≥1.2; p-value≤0.05) were identified using miRNet (Fan et al., 2016). Proteins corresponding these genes were subsequently checked for and identified in the list of proteins identified in the global proteomics analysis. Potential miRNA target genes were identified when the direction of change in the abundance of a miRNA was opposite that of a protein encoded by its regulated gene.

miR-Omics Statistical Analysis

In this multi-omic analysis of two interdependent and median values of the PoMS scales that have significant differences pre and post work shift was analyzed using a published application (Lim et al., 2017) of the Wilcoxon rank-sum test with a false discovery rate (q) of ≤0.05 being used as the benchmark for identifying candidate increased TMD/fatigue biomarkers. miRNAs satisfying this significance criterion were classified as candidate fatigue biomarkers regardless of whether they are more or less abundant post-work shift in individuals that reported increased fatigue/TMD score. Both miRNA and proteomics data for the discovery group subjects were analyzed using a variation (Suh et al., 2015) of the above application of the Wilcoxon rank-sum test to identify candidate salivary EV protein and miRNA biomarkers. A false discovery rate (q) of ≤0.05 was used as the candidate biomarker classification criterion.

The study design and flow scheme, including enrollment, saliva collection and PoMS assessment pre- and post-work shift, calculation of TMD and subscales scores for separation into Test, Discovery and Validation groups, and exosome isolation and analyses is shown in FIG. 1.

### Results

POMS Analysis and Segregation into Study Groups

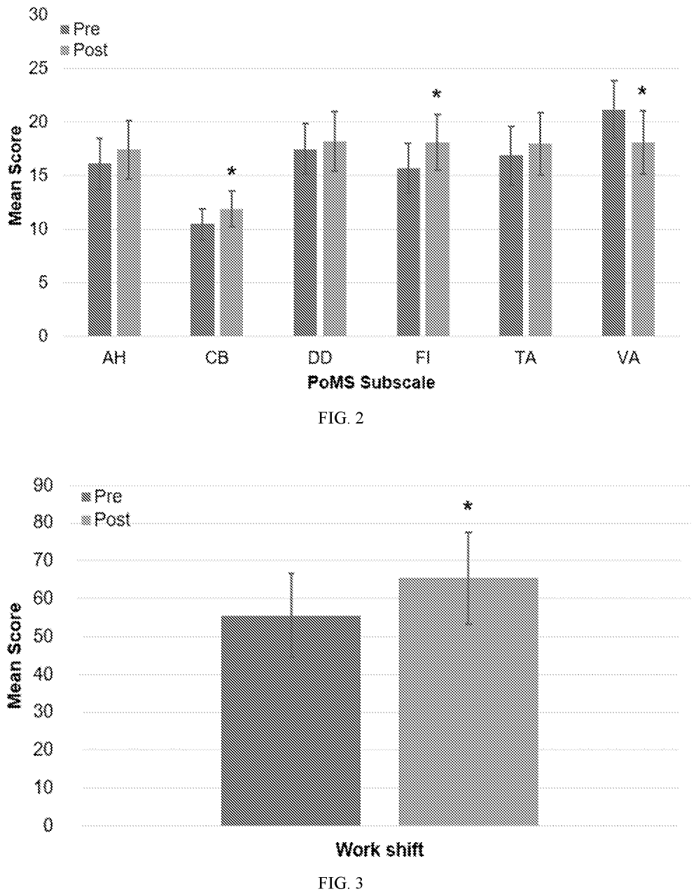

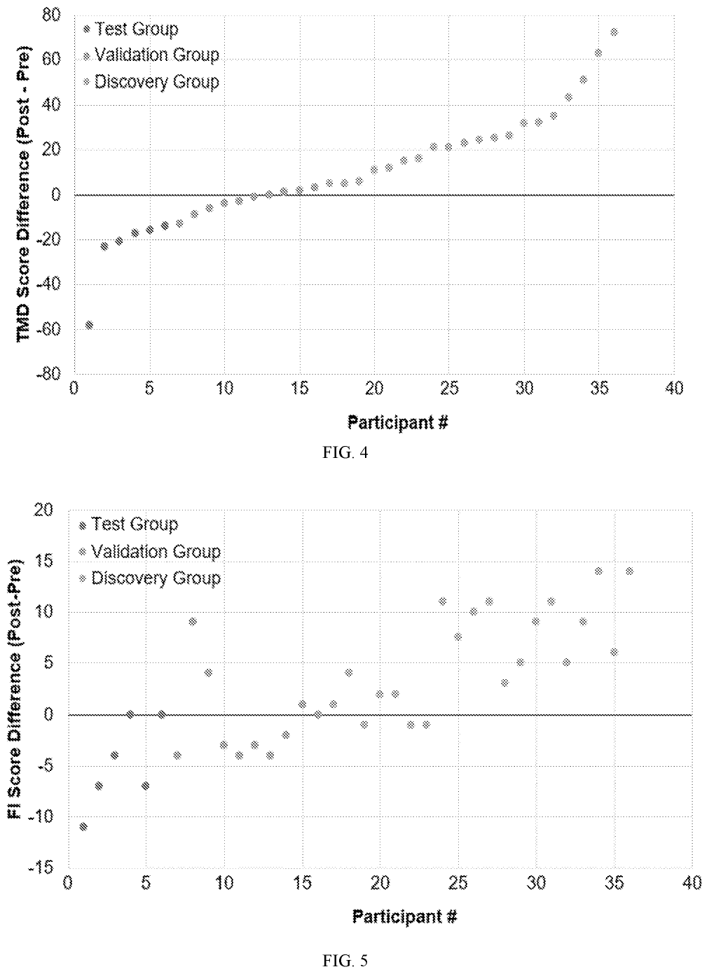

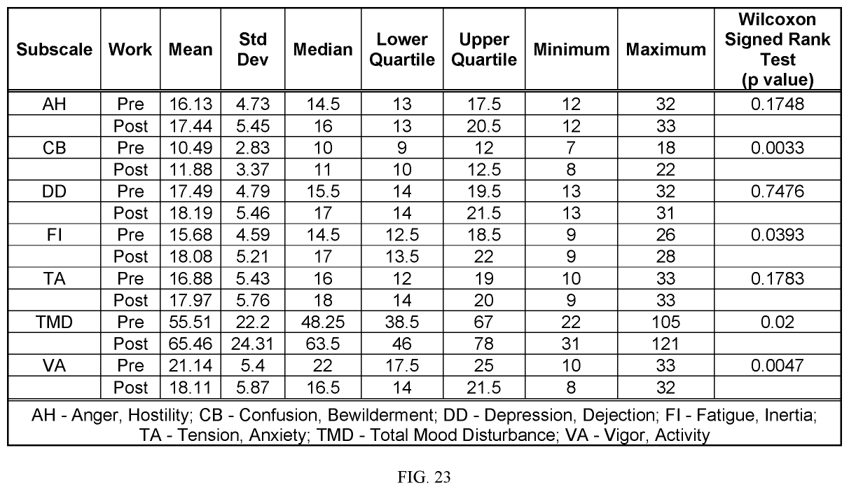

Participant answers on the PoMS questionnaire were used to calculate pre- and post-work shift TMD score. Each of the mood state subscales-TA, DD, AH, FI, CB, and VA contributed to the TMD score (FIG. 3, FIG. 23), with only the VA being subtracted from the total of the others because increased vigor and activity are associated with an improved, rather than worsening, mood. Therefore, an increase in TMD indicated a decline in mood states post-shift. Significantly increased scores were observed for CB and FI; and a decreased score for VA (FIG. 23, FIG. 2). The TMD score also significantly increased from pre-shift (53.07±20.21) to post-shift (65.99±24.83) (p<0.05), indicating that the mood of most participants worsened after the shift (FIG. 3). Not all participants recorded a positive TMD. A decrease in TMD score was observed for 12 of the 36 (33%) participants, indicative of elevated mood post-shift (FIG. 4).

As described herein, TMD score was subsequently used to segregate saliva samples into 3 groups: Test, Discovery and Validation (FIG. 4).

The purpose of the Test group was to establish the validity of analytic methods using 6 participants with a negative TMD difference (improved mood) that would not be predicted to biomarkers associated with mood disturbance. The Discovery group focused on participants with self-reported increased fatigue (FIG. 5) and decline in mood and consisted of the 20 individuals with the largest increase in TMD. The Discovery group would be expected to exhibit physiological changes associated with fatigue and an increased risk for CF. The Validation group included participants with only slightly positive and negative changes in TMD and FI, whose saliva samples underwent proteomics and qPCR analysis. Select proteins and miRNAs identified from the larger Discovery group analysis were measured and any correlation to PoMS subscales were determined.

### Multi-Omics Analysis of Test Group Saliva Exosomes

### Quantitative Global Proteomics on Salivary Exosomes

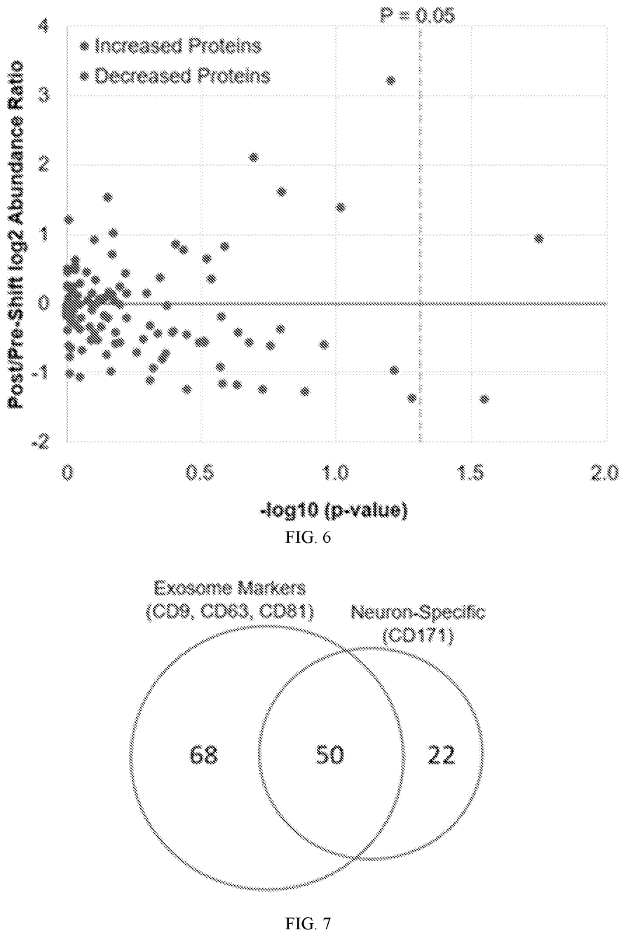

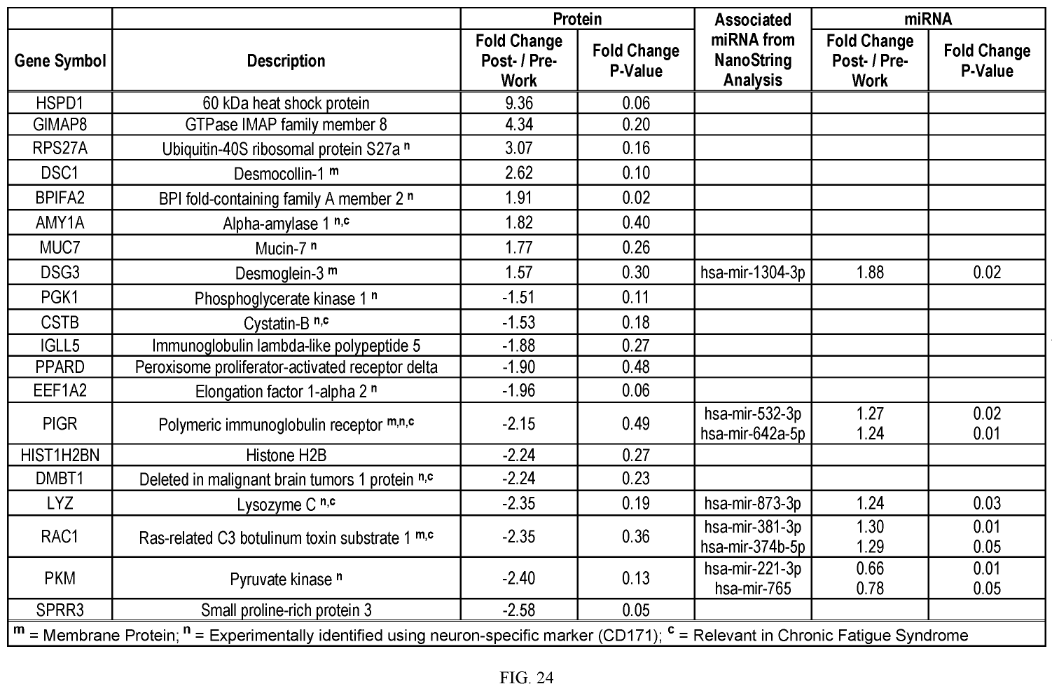

Bottom-up proteomic analysis identified a total of 121 unique proteins in exosomes enriched from the saliva of Test group participants. Reporter ion-based quantification of proteins using Tandem Mass Tags (TMT) revealed quantifiable differences in the abundance of 98 proteins extracted from the salivary exosomes of residents before and after 12-hour work shifts (FIG. 6). While changes in the abundance of only one protein, BPI fold-containing family A member 2 (BPIFA2; fold change=1.91, p=0.02) was statistically significant, many other proteins showed trends and were close to reaching significance (p≤0.05; FIG. 24). GO classifications revealed an abundance of exosome-associated proteins, including 4 altered membrane bound proteins (FIG. 24).

### Verification of Protein Measurements in Neuron-Derived Exosomes by Targeted MS

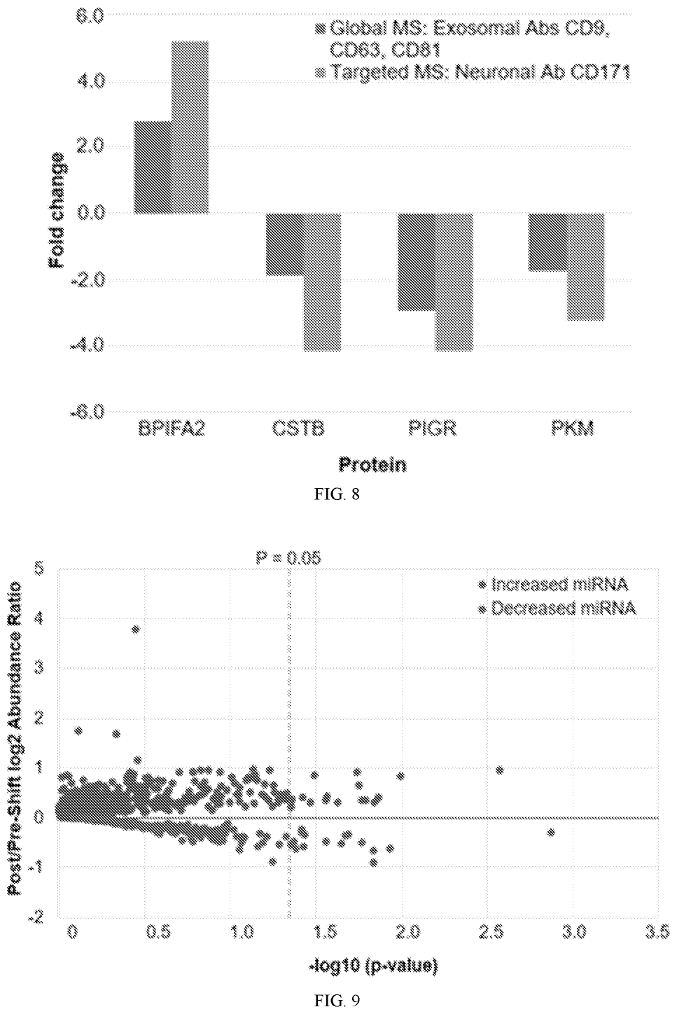

Many of the proteins identified from the immunoprecipitation using exosomal cell surface markers CD9, CD63, CD81 (FIG. 24) were subsequently identified using targeted MS for neuron-derived exosomes after isolation of salivary exosomes using neuron-cell surface marker (CD171) (FIG. 7). The targeted MS was performed for proteins with abundant peptides that contained no post-translational modifications. Four proteins—BPIFA2, CSTB, PIGR, and PKM—in neuron-derived exosomes from a single participant in the Test group correlated with global proteomics used for exosomes isolated using pan-exosomal markers, but resulted in an increase in fold change in the targeted MS as compared to global proteomics (FIG. 8).

### MicroRNA Analysis Using NanoString

The NanoString platform was used to determine the abundance of a panel of 800 biologically relevant miRNAs. While not all the miRNA species were quantifiable above background in salivary exosomes, the analysis revealed 34 miRNAs to be significantly changed in between pre- and post-work shift (absolute fold change ≥1.2, p≤0.05) (FIG. 9). Several of the significantly altered miRNA were found to target genes encoding proteins that were also determined to change in abundance between pre- and post-work shifts (FIG. 24). An inverse relationship between some identified miRNAs and their associated protein were observed.

Verification of miRNA Measurements Using qPCR

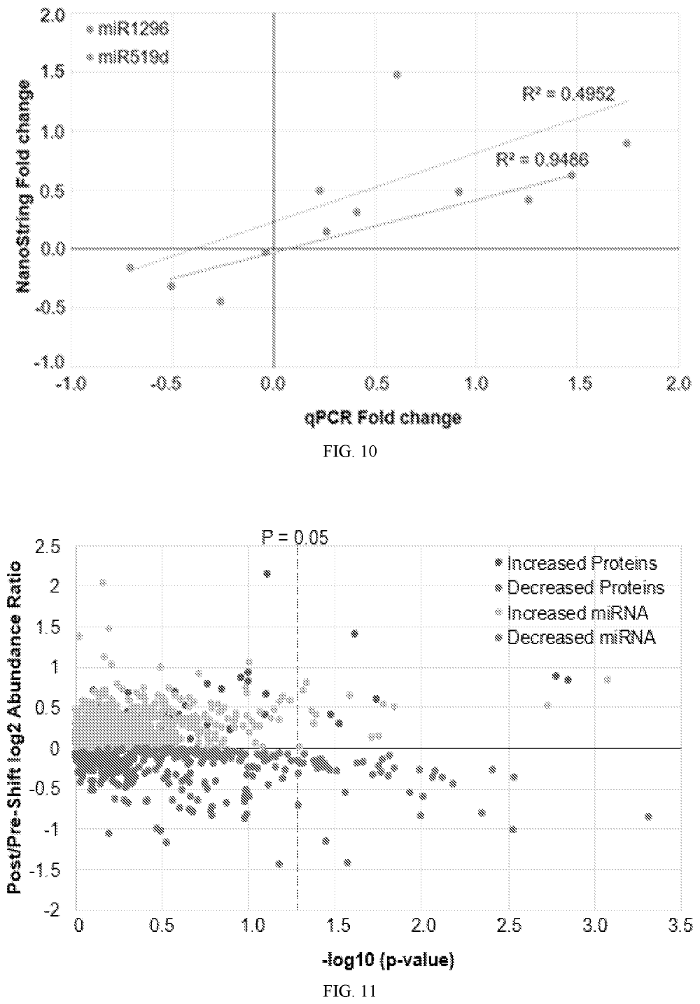

NanoString abundance measurements were validated for two select miRNAs (miR1296-3p and miR519d-3p) using qPCR. Values of % change between pre- and post-work shift for the miRNAs in the 6 Test group participants show that measurements made using the NanoString platform were qualitatively verified by qPCR (FIG. 10). This result provided the reassurance of the reliability of NanoString miRNA abundance measurements needed for subsequent analysis of the Discovery group saliva samples.

### Biomarker Identification in Discovery Group Saliva

Increased Identification of Significantly Altered Protein and miRNA

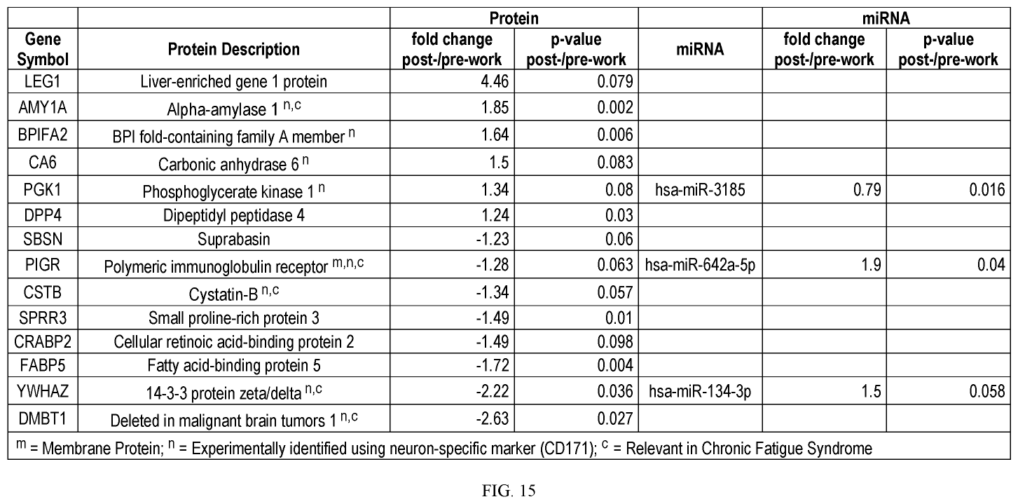

Quantitative bottom-up proteomics analyses on the salivary exosomes from the larger Discovery group (n=20) resulted in the identification of an increased number of proteins when compared to the Test group. Among the 309 protein quantified, the abundance of 7 of these proteins was determined to be significantly altered between pre- and post-work shift (absolute fold change ≥1.2 with a p-value≤0.05), and 7 additional proteins displayed trends that were close to reaching statistical significance (FIG. 11). All of proteins that exhibited a difference in absolute fold change ≥1.2 with a p-value ≤0.1 and their associated miRNA are displayed in FIG. 15.

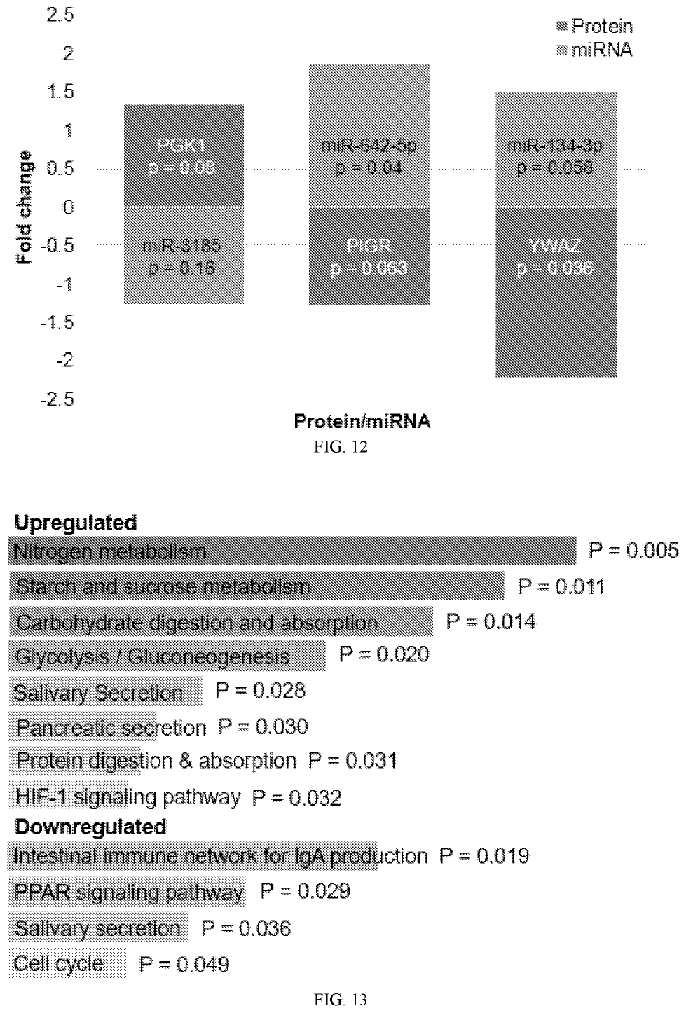

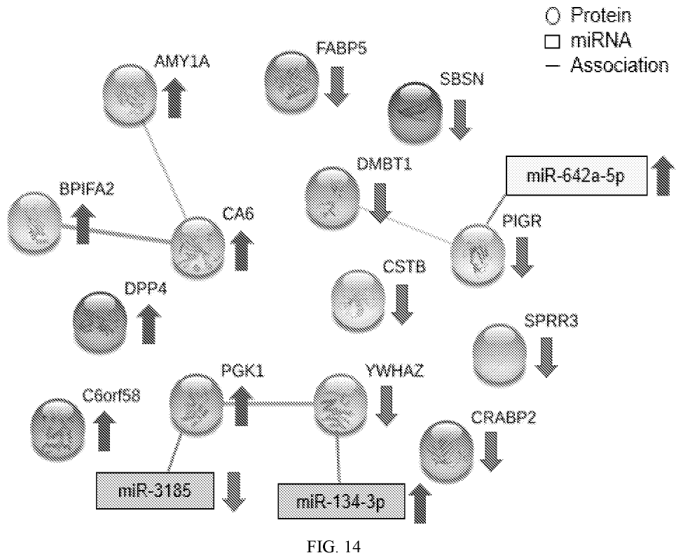

Additionally, Nanostring miRNA analysis showed 20 miRNAs to be significantly altered as well (FIG. 11). As in the Test group, the abundance of several miRNAs, miR-3185, miR-642-5p, miR-134-3p, were found to inversely correlate with the protein encoded by one of their target genes (FIG. 12). This relationship highlights three potential protein (Phosphoglycerate Kinase, gene PGK1; Polymeric Immunoglobulin Receptor, gene PIGR; and Tryptophan 5-Monooxygenase Activation Protein Zeta, gene YWHAZ) and miRNA (miR-3185, miR-642-5p, miR-134-3p) biomarkers to be assessed in Validation group saliva. Additional miRNAs assessed in the Validation group were selected based on their correlation with changes with PoMS TMD or FI subscale. Additional bioinformatic approaches including gene set enrichment, GO classification and pathway analysis (FIG. 13) and functional protein network analysis (FIG. 14) were utilized to elucidate the potential biological roles of the identified proteins in fatigue.

### Confirmation of Biomarkers and Fold Change in Validation Group Salivary Exosomes

### Validation of Protein Biomarkers

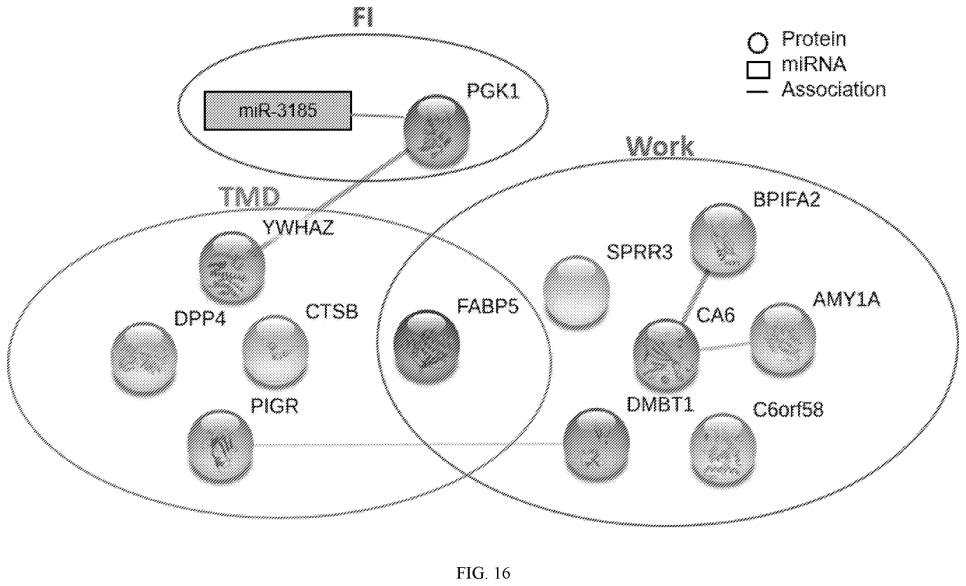

Proteomic analysis of salivary exosomes from Validation group participants confirmed the presence of 12 of the 14 candidate biomarkers identified in the Discovery group (FIG. 15, FIG. 16). As observed in the Discovery group, the mean fold-change in the abundances of 5 proteins CSTB, DDP4, FABP5, PIGR and YWAZ maintained an inverse relationship with changes in TMD score. However, that none of these changes were statistically significant (p<0.05). The abundance of 6 proteins identified in the Validation group-(DPP4, BPIFA2, CA6, AMY1A, LEG1, and DMBT1) did not correlate with changes in TMD or FI score, but exhibited mean fold-changes similar to what was observed in the Discovery group. One protein, PGK1, identified in the Discovery group was also identified in the Validation group and its fold change showed a positive but weak correlation with changes in FI score (FIG. 17).

Validation of miRNA Biomarkers

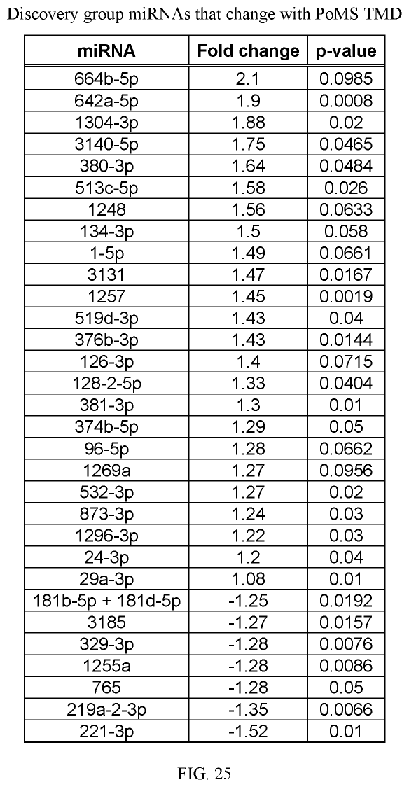

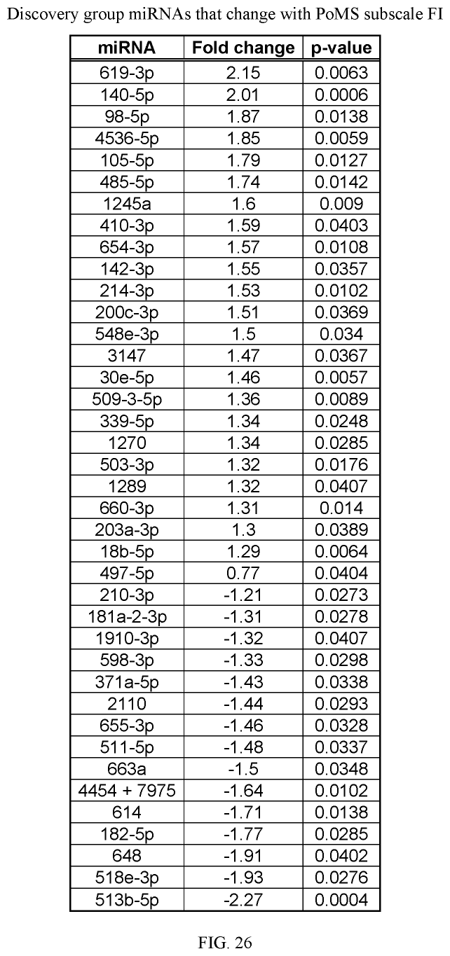

The Validation Group samples were assessed for a total of 13 miRNAs using qPCR. These miRNAs were chosen based on Discovery group NanoString results which showed either large or highly significant changes in their levels with either pre-shift/post-shift or change with PoMS subscales TMD or FI (FIG. 25 and FIG. 26), specifically miRNAs miR3185, miR29, miR1296, miR182, miR614, miR4536, miR140, miR1257, miR518e, miR105, miR126, miR642a, and miR134. The abundance of one miRNA in the validation group, miR3185, was found to correlate with PoMS FI subscale score differences (FIG. 18).

PGK1 Protein and miR3185 in Saliva as Potential Biomarkers of Fatigue

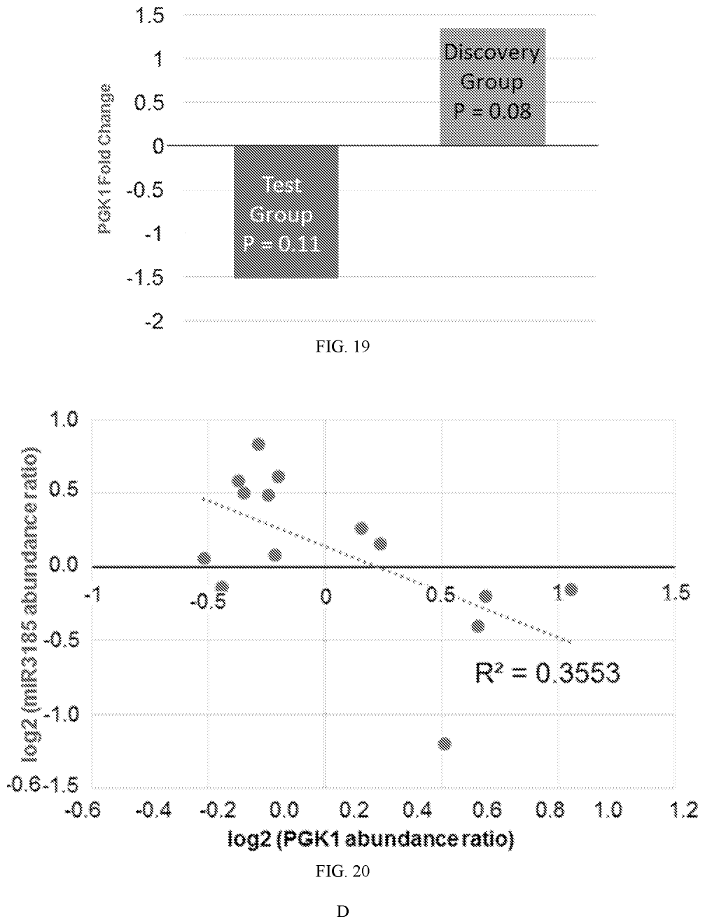

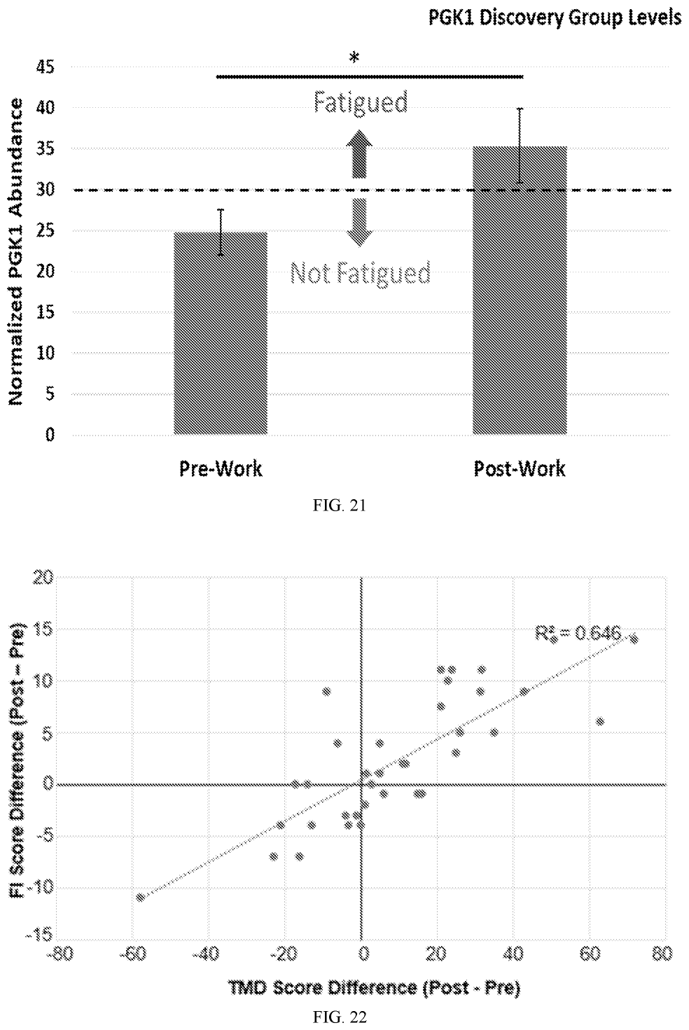

Further confirmation that PGK1 may be a promising biomarker of cognitive fatigue in saliva is seen when its fold change was evaluated in the Test group, where the residents had little fatigue compared, to the Discovery group where the Residents were more fatigued. An increased level of the PGK1 enzyme in saliva (FIG. 19) was seen in the Discovery compared to the Test group and an inverse correlation to miR3185 (FIG. 20).

### REFERENCES

The following references are herein incorporated by reference in their entirety with the exception that, should the scope and meaning of a term conflict with a definition explicitly set forth herein, the definition explicitly set forth herein controls:
Albrecht, R. R. & Ewing, S. J. (1989). Standardizing the administration of the Profile of Mood States (POMS): development of alternative word lists. J. Pers Assess, 53, 31-39.
Barber, et al. (2020) AMY1 Gene Copy Number Correlates With Glucose Absorption and Visceral Fat Volume, but Not with Insulin Resistance. J Clin Endocrinol Metab., 105 (10), dgaa473.
Balasubramanian, et al. (2015) Alterations in Salivary Proteome following Single Twenty-Minute Session of Yogic Breathing. Evid Based Complement Alternat Med. 2015:376029.
Bermejo-Pareja, et al. (2010). Saliva levels of Abeta1-42 as potential biomarker of Alzheimer's disease: a pilot study. BMC Neurol., 10, 108.
Boyd, et al. (2017). Bioenergetic status modulates motor neuron vulnerability and pathogenesis in a zebrafish model of spinal muscular atrophy. PLOS Genet, 13 (4), e1006744.
Carroll, et al. (2016). Partial sleep deprivation activates the DNA damage response (DDR) and the senescence-associated secretory phenotype (SASP) in aged adult humans. Brain Behav Immun, 51, 223-9.
Casado, B. & Baraniuk, J. N. (2009) Abstract: Decreased Mucosal Protein Secretion in the Nonallergic Rhinitis of Chronic Fatigue Syndrome. J Allergy Clin Immunol, 123 (2), S260, https://doi.org/10.1016/j.jaci.2008.12.1006.
Chen, et al. (2013). Enrichr: interactive and collaborative HTML5 gene list enrichment analysis tool. BMC bioinformatics, 14, 128. https://doi.org/10.1186/1471-2105-14-128.
Cristofaro (2015) Chronic Fatigue Syndrome: A Proteomic Approach, Ph.D. Thesis, Università di Pisa.
Ciregia, et al. (2013). A multidisciplinary approach to study a couple of monozygotic twins discordant for the chronic fatigue syndrome: a focus on potential salivary biomarkers. J. Transl. Med., 11, 243-257.
Cocucci, E. & Meldolesi, J. (2015). Ectosomes and exosomes: shedding the confusion between extracellular vesicles. Trends Cell Biol, 25, 364-72.
David, et al. (2015). Clinical Severity of PGK1 Deficiency Due To a Novel p.E120K Substitution Is Exacerbated by Co-inheritance of a Subclinical Translocation t(3;14)(q26.33;q12), Disrupting NUBPL Gene. JIMD reports, 23, 55-65.
Dominy, et al. (2014). Proteomic analysis of saliva in HIV-positive heroin addicts reveals proteins correlated with cognition. PLOS One, 9 (4), e89366.
Du, et al. (2013) Novel microRNA families expanded in the human genome. BMC Genomics, 14:98.
Fan, et al. (2016). miRNet-dissecting miRNA-target interactions and functional associations through network-based visual analysis. Nucleic acids research, 44 (W1), W135-W141.
Ferreira, J. N. & Hoffman, M. P. (2013). Interactions between developing nerves and salivary glands. Organogenesis, 9 (3), 199-205.
Fiandaca, et al. (2015). Identification of preclinical Alzheimer's disease by a profile of pathogenic proteins in neurally derived blood exosomes: A case-control study. Alzheimers Dement., 11, 600-7.
Fogt, et al. (2010). A comparison of cognitive performance decreases during acute, progressive fatigue arising from different concurrent stressors. Mil Med. 175 (12), 939-44.
Ghosh, et al. (2014). Rapid isolation of extracellular vesicles from cell culture and biological fluids using a synthetic peptide with specific affinity for heat shock proteins. PLOS One, 9 (10), e110443.
Han, et al. (2020) Identification of the miRNA-3185/CYP4A11 axis in cardiac tissue as a biomarker for mechanical asphyxia. Forensic Sci Int, 311, 110293.
Heinzelman, et al. (2019). Shotgun Proteomic Profiling of Bloodborne Nanoscale Extracellular Vesicles. Methods Mol Biol., 1897, 403-416.
Heuchert, J. P. & McNair, D. M. (2012) Profile of Mood States 2. Multi-Health Systems. APA Psych Tests database. https://psycnet.apa.org/record/9999-05057-000?doi=1.
Holtzer, et al. (2011). Cognitive fatigue defined in the context of attention networks. Neuropsychology, development, and cognition. Aging, Neuropsych Cognition, 18 (1), 108-128.
Hudson, et al. (2020) Sleep deprivation, vigilant attention, and brain function: a review. Neuropsychopharmacology, 45 (1), 21-30.
Irwin, et al. (2006). Sleep deprivation and activation of morning levels of cellular and genomic markers of inflammation. Arch Intern Med, 166, 1756-62.
Kanehisa, M. & Goto, S. (2000). KEGG: kyoto encyclopedia of genes and genomes. Nucleic Acids Res., 28 (1), 27-30.
Kimura, et al. (2018) A rat model of saliva secretory immunoglobulin: a suppression caused by intense exercise. Scand J Med Sci Sports 18 (3): 367-372.
Lim, et al. (2015). Comprehensive miRNA sequence analysis reveals survival differences in diffuse large B-cell lymphoma patients. Genome Biol., 16, 18.
Lin, et al. (2014). Review of the Profile of Mood States 2nd Edition (POMS 2). J Psychoeducational Assess., 32, 273-277.
Lorist, et al. (2005) Impaired cognitive control and reduced cingulate activity during mental fatigue. Cogn Brain Res, 24 (2), 199-205.
Love, K. M., & Liu, Z. (2021) DPP4 Activity, Hyperinsulinemia, and Atherosclerosis. J Clin Endocrinol Metab, 106 (6), 1553-1565.
Maes, et al. (2012). Evidence for inflammation and activation of cell-mediated immunity in myalgic encephalomyelitis/chronic fatigue syndrome (ME/CFS): increased interleukin-1, tumor necrosis factor-α, PMN-elastase, lysozyme and neopterin. J Affect Disord 136, 933-939.
McNair, et al. (1971). Manual for the Profile of Mood States. San Diego: Educational and Industrial Testing Service.
McNair, et al. (1992). Profile of mood states. Educational and industrial testing service.
Meeusen, et al. (2021) Endurance exercise-induced mental fatigue and the brain. Exp Physiol, 106 (12), 2294-2298.
Mitra, et al. (2017). Plasma micro-RNA biomarkers for diagnosis and prognosis after traumatic brain injury: A pilot study. J Clin Neurosci, 38, 37-42.
Multhoff, G., & Hightower, L. E. (2011). Distinguishing integral and receptor-bound heat shock protein 70 (Hsp70) on the cell surface by Hsp70-specific antibodies. Cell Stress Chaperones, 16, 251-5.
Nagashima, et al. (2014). Hepatitis E virus egress depends on the exosomal pathway, with secretory exosomes derived from multivesicular bodies. J Gen Virol, 95, 2166-75.
Nie, et al. (2020) O-GlcNAcylation of PGK1 coordinates glycolysis and TCA cycle to promote tumor growth. Nature Commun, 11 (36).
Pascut, et al. (2020). Weighted miRNA co-expression networks analysis identifies circulating miRNA predicting overall survival in hepatocellular carcinoma patients. Sci Rep 10, 18967, DOI: 10.1038/s41598-020-75945-2.
Pietro, et al. (2018). Salivary MicroRNAS: Diagnostic Markers of Mild Traumatic Brain Injury in Contact-Sport. Front Molec Neurosci, 290, 1-13.
Pihur, et al. (2011). Meta analysis of Chronic Fatigue Syndrome through integration of clinical, gene expression, SNP and proteomic data. Bioinformation, 6 (3), 120-124.
Rappsilber, et al. (2007). Protocol for micro-purification, enrichment, pre-fractionation and storage of peptides for proteomics using StageTips. Nature protocols, 2 (8), 1896-1906.
Savitski, et al. (2013). Measuring and managing ratio compression for accurate iTRAQ/TMT quantification. J Proteome Res, 12 (8), 3586-3598.
Shi, et al. (2011). Salivary tau species are potential biomarkers of Alzheimer's disease. J Alzheimers Dis . . . 27, 299-305.
Suh, et al. (2015). Quantitative Differences in the Urinary Proteome of Siblings Discordant for Type 1 Diabetes Include Lysosomal Enzymes. J Proteome Res., 14, 3123-35.
Szklarczyk, et al. (2019). STRING v11: protein-protein association networks with increased coverage, supporting functional discovery in genome-wide experimental datasets. Nucleic Acids Res, 47 (D1), D607-D613.
Vissing, et al. (2018) Level of residual enzyme activity modulates the phenotype in phosphoglycerate kinase deficiency. Neurology, 91 (11).
Yáñez-Mó, et al. (2015) Biological properties of extracellular vesicles and their physiological functions. J Extracell Vesicles, 4, 27066.
Zhu, et al. (2012). Sleep disturbance induces neuroinflammation and impairment of learning and memory. Neurobiol Dis, 48, 348-55.

All scientific and technical terms used in this application have meanings commonly used in the art unless otherwise specified.

All scientific and technical terms used in this application have meanings commonly used in the art unless otherwise specified.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of a given ingredient in a composition, means that the composition may include additional ingredients so long as the additional ingredients do not adversely impact the activity, e.g., biological or pharmaceutical function, of the given ingredient.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus. Except when specifically indicated, peptides are indicated with the N-terminus on the left and the sequences are written from the N-terminus to the C-terminus. Similarly, except when specifically indicated, nucleic acid sequences are indicated with 5′ end on the left and the sequences are written from 5′ to 3′.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0±, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of preparing an exosome sample for measuring the amount of a biomarker, preferably PGK1 and/or miR3185, therein, which comprises obtaining a saliva sample from one or more subjects, adding a protease inhibitor to the saliva sample, removing solids in the saliva sample to obtain a supernatant, and isolating exosomes present in the supernatant on a substrate surface.

2. A method of measuring or evaluating expression levels of a biomarker, preferably PGK1 and/or miR3185, in an exosome sample obtained from a saliva sample, which comprises obtaining an exosome sample that has been prepared according to the method of claim 1, and measuring the amount of the biomarker.

3. (canceled)

4. A method of diagnosing a subject as suffering from cognitive fatigue, which comprises measuring the amount of a biomarker, which is PGK1 and/or miR3185, in an exosome sample obtained from a saliva sample from the subject, comparing the amount with a control, and identifying the subject as suffering from cognitive fatigue where the measured amount of PGK1 is about 1.3-1.4 fold increase and/or the amount of miR3185 is about 1.2-1.3 decrease compared to the control.

5. The diagnosis method according to claim 4, wherein the exosome sample was prepared by obtaining a saliva sample from the subject, adding a protease inhibitor to the saliva sample, removing solids in the saliva sample to obtain a supernatant, and isolating exosomes present in the supernatant on a substrate surface.

6. The diagnosis method according to claim 4, and measuring in the exosome sample one or more additional biomarkers selected from the group consisting of: LEG1, AMY1A, BPIFA2, CA6, DPP4, DMBT1, and the microRNAs selected from hsa-miR-664b-5p, hsa-miR-642a-5p, hsa-miR-1304-3p, hsa-miR-3140-5p, hsa-miR-380-3p, hsa-miR-513c-5p, hsa-miR-1248, hsa-miR-134-3p, hsa-miR-1-5p, hsa-miR-3131, hsa-miR-1257, hsa-miR-376b-3p, hsa-miR-519d-3p, hsa-miR-126-3p, hsa-miR-126-3p, hsa-miR-128-2-5p, hsa-miR-381-3p, hsa-miR-374b-5p, hsa-miR-96-5p, hsa-miR-1269a, hsa-miR-532-3p, hsa-miR-873-3p, hsa-miR-1296-3p, hsa-miR-24-3p, hsa-miR-29a-3p, hsa-miR-181b-5p, hsa-miR-181d-5pb, hsa-miR-1255a, hsa-miR-329-3p, hsa-miR-765, hsa-miR-219a-2-3p, hsa-miR-221-3p, hsa-miR-619-3p, hsa-miR-140-5p, hsa-miR-98-5p, hsa-miR-4536-5p, hsa-miR-105-5p, hsa-miR-485-5p, hsa-miR-1245a, hsa-miR-410-3p, hsa-miR-654-3p, hsa-miR-142-3p, hsa-miR-214-3p, hsa-miR-200c-3p, hsa-miR-548e-3p, hsa-miR-3147, hsa-miR-30e-5p, hsa-miR-509-3-5p, hsa-miR-1270, hsa-miR-339-5p, hsa-miR-1289, hsa-miR-503-3p, hsa-miR-660-3p, hsa-miR-203a-3p, hsa-miR-18b-5p, hsa-miR-497-5p, hsa-miR-210-3p, hsa-miR-181a-2-3p, hsa-miR-1910-3p, hsa-miR-598-3p, hsa-miR-371a-5p, hsa-miR-2110, hsa-miR-655-3p, hsa-miR-511-5p, hsa-miR-663a, hsa-miR-4454, hsa-miR-7975b, hsa-miR-614, hsa-miR-182-5p, hsa-miR-648, hsa-miR-518e-3p, and hsa-miR-513b-5p (preferably hsa-miR-518e-3p, hsa-miR-182-5p, hsa-miR-614, hsa-miR-1296-3p, hsa-miR-126-3p, hsa-miR-1257, hsa-miR-134-3p, hsa-miR-105-5p, hsa-miR-4536-5p, hsa-miR-642a-5p, and hsa-miR-140-5p).

7. The diagnosis method according to claim 6, which comprises diagnosing the subject as suffering from cognitive fatigue where:
A) the amount of LEG1, AMY1A, BPIFA2, CA6, and/or DPP4 is increased,
B) the amount of DMBT1 is decreased, or
C) both A) and B) as compared to the control.

8. The diagnosis method according to claim 6, which comprises diagnosing the subject as suffering from cognitive fatigue where:
a) the amount of hsa-miR-1296-3p, hsa-miR-126-3p, hsa-miR-1257, hsa-miR-134-3p, hsa-miR-105-5p, hsa-miR-4536-5p, hsa-miR-642a-5p, and/or hsa-miR-140-5p,
b) the amount of hsa-miR-518e-3p, hsa-miR-182-5p, and/or hsa-miR-614 is decreased, or
c) both a) and b) as compared to the control.

9. The diagnosis method according to claim 6, which comprises diagnosing the subject as suffering from cognitive fatigue where:
A)
1) the amount of LEG1, AMY1A, BPIFA2, CA6, and/or DPP4 is increased,
2) the amount of DMBT1 is decreased, or
3) both 1) and 2); and
B)
i) the amount of hsa-miR-1296-3p, hsa-miR-126-3p, hsa-miR-1257, hsa-miR-134-3p, hsa-miR-105-5p, hsa-miR-4536-5p, hsa-miR-642a-5p, and/or hsa-miR-140-5p,
ii) the amount of hsa-miR-518e-3p, hsa-miR-182-5p, and/or hsa-miR-614 is decreased, or
iii) both i) and ii)
as compared to the control.

10. The diagnosis method according to claim 4, wherein the saliva sample is obtained from the subject after deliberately and consciously performing a cognitive activity for a given period of time with or without one or more breaks.

11. The diagnosis method according to claim 10, wherein the given period of time is about 1 hour or more, about 2 hours or more, about 3 hours or more, about 4 hours or more, about 5 hours or more, about 6 hours or more, about 7 hours or more, about 8 hours or more, about 9 hours or more, about 10 hours or more, about 11 hours or more, about 12 hours or more, about 13 hours or more, about 14 hours or more, about 15 hours or more, about 16 hours or more, about 17 hours or more, about 18 hours or more, about 19 hours or more, about 20 hours or more, about 21 hours or more, about 22 hours or more, about 23 hours or more, or about 24 hours or more.

12. The diagnosis method according to claim 10, wherein each break is independently about 5 minutes to about 45 minutes with the ratio of the sum amount of the one or more breaks to the given period of time being about 1:25 to about 1:8 or less.

13. The diagnosis method according to claim 4, wherein the biomarkers that are proteins are measured via a lateral flow assay device and/or the biomarkers that are microRNAs are measured via PCR.

14. The diagnosis method according to claim 13, wherein an amount of PGK1 that is about 1.3-1.4 fold more than a given control and/or the amount of miR3185 that is about 1.2-1.3 fold less than the given control results in a positive reading for cognitive fatigue.

15. A method of evaluating expression levels of PGK1 and miR3185 in a subject, which consists of:
obtaining an exosome sample from a saliva sample from the subject,
measuring the amount of the biomarker in the exosome sample, and
optionally measuring in the exosome sample the amount of one or more additional biomarkers selected from the group consisting of: LEG1, AMY1A, BPIFA2, CA6, DPP4, DMBT1, hsa-miR-518e-3p, hsa-miR-182-5p, hsa-miR-614, hsa-miR-1296-3p, hsa-miR-126-3p, hsa-miR-1257, hsa-miR-134-3p, hsa-miR-105-5p, hsa-miR-4536-5p, hsa-miR-642a-5p, and hsa-miR-140-5p.

16. The method according to claim 2, and measuring in the exosome sample one or more additional biomarkers selected from the group consisting of: LEG1, AMY1A, BPIFA2, CA6, DPP4, DMBT1, and the microRNAs selected from hsa-miR-664b-5p, hsa-miR-642a-5p, hsa-miR-1304-3p, hsa-miR-3140-5p, hsa-miR-380-3p, hsa-miR-513c-5p, hsa-miR-1248, hsa-miR-134-3p, hsa-miR-1-5p, hsa-miR-3131, hsa-miR-1257, hsa-miR-376b-3p, hsa-miR-519d-3p, hsa-miR-126-3p, hsa-miR-126-3p, hsa-miR-128-2-5p, hsa-miR-381-3p, hsa-miR-374b-5p, hsa-miR-96-5p, hsa-miR-1269a, hsa-miR-532-3p, hsa-miR-873-3p, hsa-miR-1296-3p, hsa-miR-24-3p, hsa-miR-29a-3p, hsa-miR-181b-5p, hsa-miR-181d-5pb, hsa-miR-1255a, hsa-miR-329-3p, hsa-miR-765, hsa-miR-219a-2-3p, hsa-miR-221-3p, hsa-miR-619-3p, hsa-miR-140-5p, hsa-miR-98-5p, hsa-miR-4536-5p, hsa-miR-105-5p, hsa-miR-485-5p, hsa-miR-1245a, hsa-miR-410-3p, hsa-miR-654-3p, hsa-miR-142-3p, hsa-miR-214-3p, hsa-miR-200c-3p, hsa-miR-548e-3p, hsa-miR-3147, hsa-miR-30e-5p, hsa-miR-509-3-5p, hsa-miR-1270, hsa-miR-339-5p, hsa-miR-1289, hsa-miR-503-3p, hsa-miR-660-3p, hsa-miR-203a-3p, hsa-miR-18b-5p, hsa-miR-497-5p, hsa-miR-210-3p, hsa-miR-181a-2-3p, hsa-miR-1910-3p, hsa-miR-598-3p, hsa-miR-371a-5p, hsa-miR-2110, hsa-miR-655-3p, hsa-miR-511-5p, hsa-miR-663a, hsa-miR-4454, hsa-miR-7975b, hsa-miR-614, hsa-miR-182-5p, hsa-miR-648, hsa-miR-518e-3p, and hsa-miR-513b-5p (preferably hsa-miR-518e-3p, hsa-miR-182-5p, hsa-miR-614, hsa-miR-1296-3p, hsa-miR-126-3p, hsa-miR-1257, hsa-miR-134-3p, hsa-miR-105-5p, hsa-miR-4536-5p, hsa-miR-642a-5p, and hsa-miR-140-5p).

17. The method according to claim 2, wherein the saliva sample is obtained from the subject or subjects after deliberately and consciously performing a cognitive activity for a given period of time with or without one or more breaks.

18. The method according to claim 17, wherein the given period of time is about 1 hour or more, about 2 hours or more, about 3 hours or more, about 4 hours or more, about 5 hours or more, about 6 hours or more, about 7 hours or more, about 8 hours or more, about 9 hours or more, about 10 hours or more, about 11 hours or more, about 12 hours or more, about 13 hours or more, about 14 hours or more, about 15 hours or more, about 16 hours or more, about 17 hours or more, about 18 hours or more, about 19 hours or more, about 20 hours or more, about 21 hours or more, about 22 hours or more, about 23 hours or more, or about 24 hours or more.

19. The method according to claim 17, wherein each break is independently about 5 minutes to about 45 minutes with the ratio of the sum amount of the one or more breaks to the given period of time being about 1:25 to about 1:8 or less.
